(19) United States
(12) Patent Application Publication
(10) Pub. No.: US 2026/0027159 A1
(43) Pub. Date: Jan. 29, 2026

(54) COMPOSITION FOR PREVENTION OR TREATMENT OF NEUROLOGICAL DISEASE COMPRISING: SCHWANN CELL PRECURSOR (SCP) OR SCHWANN CELL (SC) DIFFERENTIATED THEREFROM; AND NATURAL KILLER (NK) CELL

(71) Applicant: KOREA RESEARCH INSTITUTE OF BIOSCIENCE AND BIOTECHNOLOGY, Daejeon (KR)

(72) Inventors: Yee Sook CHO, Daejeon (KR); Han-Seop KIM, Daejeon (KR); Jae Yun KIM, Daejeon (KR); Binna SEOL, Daejeon (KR)

(73) Assignee: KOREA RESEARCH INSTITUTE OF BIOSCIENCE AND BIOTECHNOLOGY, Daejeon (KR)

(21) Appl. No.: 19/346,862

(22) Filed: Oct. 1, 2025

Related U.S. Application Data
(63) Continuation-in-part of application No. PCT/KR2024/014128, filed on Sep. 19, 2024.

(30) Foreign Application Priority Data
Dec. 7, 2023 (KR) 10-2023-0176906

(51) Int. Cl. A61K 35/30; A61K 35/17; A61P 25/02; A61P 25/16; C12N 5/0783; C12N 5/079

(52) U.S. Cl. CPC A61K 35/30; A61K 35/17; A61P 25/02; A61P 25/16; C12N 5/0622; C12N 5/0646; C12N 2501/115; C12N 2501/13; C12N 2501/135; C12N 2501/999

(57) ABSTRACT

The present invention relates to a pharmaceutical composition and a cell therapy agent for preventing or treating a neurological disease, comprising Schwann cell precursors (SCPs) prepared from pluripotent stem cells (PSCs) or somatic cells, or Schwann cells (SCs) differentiated therefrom; and natural killer (NK) cells as active ingredients. Specifically, the present invention is characterized in that the SCPs express one or more selected from the group consisting of GAP43, SOX10, IGFBP2, and a combination thereof; the SCs express one or more selected from the group consisting of S100B, SOX10, and a combination thereof; and the NK cells express one or more selected from the group consisting of CD56⁺, CD16⁺, and a combination thereof.

### CROSS-REFERENCE TO RELATED APPLICATIONS

This application is a Continuation-in-Part of International Application No. PCT/KR2024/014128 filed Sep. 19, 2024, claiming priority based on Korean Patent Application No. 10-2023-0176906 filed Dec. 7, 2023, the disclosure of which is incorporated herein by reference in its entirety.

### INCORPORATION BY REFERENCE OF SEQUENCE LISTING

The instant application contains a Sequence Listing which has been filed electronically in xml format and is hereby incorporated by reference in its entirety. Said xml file, created on Oct. 1, 2025, is named Q313985_sequence listing as filed.xml and is 36,373 bytes in size.

### TECHNICAL FIELD

The present invention relates to a pharmaceutical composition and a cell therapy agent for preventing or treating a neurological disease, comprising: Schwann cell precursors (SCPs) prepared from pluripotent stem cells (PSCs) or somatic cells, or Schwann cells (SCs) differentiated therefrom; and natural killer (NK) cells as active ingredients. Specifically, the present invention is characterized in that: the SCPs express one or more selected from the group consisting of GAP43, SOX10, IGFBP2,and a combination thereof; the SCs express one or more selected from the group consisting of S100B, SOX10, and a combination thereof; and the NK cells express one or more selected from the group consisting of CD56⁺, CD16⁺, and a combination thereof.

### BACKGROUND ART

Schwann cells (SCs) are essential neuroglial cells in the peripheral nervous system (PNS) that play a central role in supporting neurons and promoting nerve repair. Schwann cells are responsible for forming myelin that insulates nerve fibers in the PNS and ensures rapid transmission of nerve impulses. Myelination by Schwann cells is essential not only for normal nerve function but also for the repair and regeneration of damaged nerves. In addition to their role in myelination, Schwann cells secrete various neurotrophic factors, such as brain-derived neurotrophic factor (BDNF), glial cell-derived neurotrophic factor (GDNF), nerve growth factor (NGF), and neurotrophin-3 (NT-3), which promote neuronal survival, enhance axonal growth, and support nerve regeneration after injury. Schwann cells also produce components of the extracellular matrix that provide a scaffold promoting axonal regrowth and regeneration, thereby creating an environment conducive to nerve repair.

Due to the inherent characteristics of human Schwann cells, they are highly useful for new therapeutic developments such as disease modeling, drug discovery, and cell therapy; however, it is difficult to secure a sufficient number of functional cells. In the case of primary cultured Schwann cells derived from human tissue, the isolation efficiency and culture purity vary depending on the condition of the nerve biopsy, and harvesting requires an invasive procedure accompanied by risks such as pain and potential nerve damage. In ex vivo culture, Schwann cells exhibit low proliferative ability, making it difficult to expand them to a sufficient cell number, and as culture time progresses, increases in fibroblast contamination may result in decreased Schwann cell purity and deterioration in quality. It is important that the cultured Schwann cells maintain their original characteristics (such as myelinating ability and secretion of neurotrophic factors), but long-term culture and expansion ex vivo can lead to loss of Schwann cell characteristics and functions.

Alternatively, active research and development is underway to optimize and scale up the production of Schwann cells from stem cells. In particular, human pluripotent stem cells (PSCs), including human embryonic stem cells (ESCs) and human induced pluripotent stem cells (iPSCs), have excellent proliferative and differentiating abilities and are recognized as important resources for Schwann cell differentiation and production. During development, Schwann cells exist in various forms, such as (1) neural crest (stem) cells [NC(S) Cs], (2) Schwann cell precursors (SCPs), (3) immature non-myelinating Schwann cells, and (4) mature myelinating Schwann cells. In general, methods for obtaining Schwann cells from PSCs involve first differentiating PSCs into multipotent neural crest stem cells (NCSCs), which are developmental precursors of Schwann cells, and then re-differentiating the NCSCs into Schwann cells. However, problems associated with differentiating PSCs into Schwann cells through NCSCs include (1) complex and time-consuming differentiation process, (2) low productivity and purity, and (3) poor biological function and therapeutic performance.

Distinctively, Schwann cell precursors (SCPs) exist as an intermediate cell type, distinguished from neural crest cells at an early developmental stage and non-myelinating Schwann cells. SCPs obtained from PSCs can be proliferatively cultured and are capable of directly producing Schwann cells within a short period, highlighting their importance as an optimal Schwann cell source. Accordingly, the present technique produced Schwann cell precursors (PSC-SCPs) and Schwann cells (PSC-SCP-SCs) from PSCs, and analyzed their effects in targeting the treatment of nerve injuries/disorders.

Meanwhile, natural killer (NK) cells are a type of lymphoid blood cell that play an important role in both innate and adaptive immune responses. In particular, without the need to recognize specific antigens, they detect abnormal proteins on the cell surface or the reduction of major histocompatibility complex (MHC) class I molecules, and thereby identify and promptly eliminate abnormal cells that cause diseases, such as cancer cells and cells infected with viruses, bacteria, fungi, or parasites. Thus, NK cells have become an important target for therapeutic development against various diseases.

NK cells can promote nerve repair and regeneration by eliminating damaged nerves, influence other immune cells such as microglia and T cells to suppress neuroinflammation and the induction of autoimmune diseases, and eliminate nerve cells infected by viruses, etc. to inhibit the spread of infection within neural tissue. Accordingly, the potential utility of NK cells has been highlighted in the treatment of various neurological diseases, including nerve injury disorders and neurodegenerative diseases such as Alzheimer's disease and Parkinson's disease; however, research and technological development regarding their direct role and potential therapeutic effects remain insufficient.

Recently, with the advancement of direct reprogramming technology, active development has been underway for techniques that directly produce functional cells with high clinical utility without undergoing production processes of stem cells such as PSCs. Functional cells produced via direct reprogramming have been recognized for their technical advantages, including reduced risk of epigenetic remodeling and tumor formation and simplification of the cell production process, thereby facilitating improvement in safety, reliability, and efficiency. Such characteristics are expected to ultimately contribute to overcoming barriers to commercialization by drastically shortening and reducing the time and cost required for therapeutic development, and thus, research and development efforts to secure raw materials for cell therapy agents targeting various diseases are continuously increasing. Accordingly, the present technology analyzed the effects of Schwann cell precursors (drSCPs) and NK (drNK) cells obtained via direct reprogramming in the treatment of nerve injuries/disorders.

To date, there have been no reports on the preventive, therapeutic, and ameliorative effects against neurological diseases of a combination of Schwann cell precursors prepared from pluripotent stem cells (PSC-SCPs) or Schwann cells differentiated therefrom (PSC-SCP-SCs), somatic cell reprogrammed Schwann cell precursors (drSCPs) or Schwann cells (drSCP-SCs) differentiated therefrom, and NK cells.

### DISCLOSURE

### Technical Problem

The present inventors have made extensive efforts to develop a rapid and highly efficient method for preparing a functionally improved neuro-injury/disorder therapeutic agent, and as a result, they have produced Schwann cell precursors capable of ex vivo proliferation via differentiation culture from human pluripotent stem cells or via direct reprogramming culture from somatic cells; and confirmed that the induction of the Schwann cell precursors to differentiate into Schwann cells enables production of human Schwann cells with enhanced functionality both in vivo and ex vivo, under a shortened period of time with improved production efficiency. In addition, the present inventors have confirmed that a combined use thereof with human natural killer (NK) cells can be usefully employed for the prevention or treatment of a neurological disease, thereby completing the present invention.

### Technical Solution

One object of the present invention is to provide a pharmaceutical composition for preventing or treating a neurological disease, comprising Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10, and a combination thereof; and natural killer (NK) cells expressing one or more selected from the group consisting of CD56⁺, CD16⁺, and a combination thereof.

Another object of the present invention is to provide a cell therapy agent composition for preventing or treating a neurological disease, comprising Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10, and a combination thereof; and natural killer (NK) cells expressing one or more selected from the group consisting of CD56⁺, CD16⁺, and a combination thereof.

Another object of the present invention is to provide a method for preventing or treating a neurological disease, comprising administering to a non-human subject suspected of having a neurological disease, a pharmaceutical composition for preventing or treating a neurological disease comprising Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10, and a combination thereof; and natural killer (NK) cells expressing one or more selected from the group consisting of CD56⁺, CD16⁺, and a combination thereof.

Another object of the present invention is to provide a method for preventing or treating a neurological disease, comprising administering to a subject in need thereof an effective amount of Schwann cell precursors (SCPs) or Schwann cells (SCs) differentiated therefrom, and natural killer (NK) cells, wherein:
the Schwann cell precursors (SCPs) express one or more selected from the group consisting of GAP43, SOX10, IGFBP2, and a combination thereof;
the Schwann cells (SCs) express one or more selected from the group consisting of S100B, SOX10, and a combination thereof; and
the natural killer (NK) cells express one or more selected from the group consisting of CD56⁺, CD16⁺, and a combination thereof.

### Advantageous Effects

Administration of Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10, and a combination thereof; and natural killer (NK) cells expressing one or more selected from the group consisting of CD56⁺, CD16⁺, and a combination thereof of the present invention, either alone or in combination, to peripheral and central nervous system-injured disease models exhibited gene expression characteristics different from conventional Schwann cell precursors (NCSCs); a superior secreting ability of the neurotrophic factor GDNF compared to conventional NCSCs; and excellent regenerative and therapeutic effects on the damaged nervous system. Accordingly, the present invention can be usefully employed for the prevention or treatment of neurological diseases.

### BRIEF DESCRIPTION OF THE DRAWINGS

FIG. 1 is a schematic diagram of the differentiation induction process from human pluripotent stem cells (hPSCs) to Schwann cell precursors (hSCPs), and shows the development of improved Protocols 4 and 5 with the introduction of new factors, as compared with the conventional control Protocol 1. A of FIG. 1 is a schematic diagram showing the differentiation from human pluripotent stem cells (hPSCs) to Schwann cell precursors (hSCPs) according to the conventional control (Protocol 1) and the present invention (Protocols 2 to 8). B of FIG. 1 is a diagram showing different differentiation medium compositions used in Stages 1 and 2 of A of FIG. 1. C of FIG. 1 is a quantitative analysis of the expression level of the SOX10gene from total RNA of cells on day 24, according to the differentiation induction method of B of FIG. 1. D of FIG. 1 is a quantitative analysis of the gene expression levels of CD49d, ERBB3, and PLP1, additionally conducted in groups in which an increase in SOX10 was confirmed in Protocols 1, 4, 5, and 7 in C of FIG. 1. E of FIG. 1 is a result confirming the protein expression of SOX10, GAP43, and IGFBP2 in SCPs differentiated by the conventional method (Protocol 1) and novel methods (Protocols 4 and 5) involving the introduction of novel factors through immunocytochemical staining.

FIG. 2 shows the quantitative analysis of Schwann cell markers S100b, NGFR, MPZ, and EGR2 gene expression in Schwann cells differentiated from Schwann cell precursors produced using Protocols 1 and 4 of FIG. 1 (Protocol 1-SC, Protocol 4-SC) and from Schwann cells produced through differentiation induction culture from SCPs generated by somatic cell reprogramming (iSCP-SC) (A of FIG. 2). B of FIG. 2 shows the results confirming the expression of SOX10 and S100B proteins in the differentiated Schwann cells of A of FIG. 2.

FIG. 3 shows the results confirming that the expression of neurotrophic factors GDNF and IGFBP-2 is higher in the induced SCPs of the present invention than in conventional Schwann cell precursors (NCSCs). A of FIG. 3 shows the quantitative analysis of the gene expression levels of GDNF and IGFBP-2 in H9 (hPSCs), NCSCs, and SCPs, respectively, and B of FIG. 3 shows the quantitative analysis of the protein secretion levels of GDNF and IGFBP-2 in the conditioned media (CM) of NCSCs and SCPs, respectively.

FIG. 4 shows the cellular phenotypic characteristics of the specified directly reprogrammed natural killer (drNK) cells of the present invention. To compare and analyze the effect of natural killer cells targeting injured nerves of the present invention, A of FIG. 4 shows a list of four representative types of NK cells that exhibit different CD56 and CD16 expression patterns: 1. CD56^{dim}pNK, the main cell type of PBMC-derived NK cells; 2. CD56^{bright}pNK, activated by IL-2/IL-15 cytokine; 3. NK92, an immortalized NK cell line; and 4. drNK of the present invention, prepared through direct reprogramming. B of FIG. 4 shows CD56 and CD16 expression patterns of each NK cell using a flow cytometer, confirming that the four types of NK cells exhibit different characteristics. C of FIG. 4 shows a comparison of the expression of cell surface receptors related to NK cytotoxicity in CD56^{dim}pNK and drNK cells using a flow cytometer.

FIG. 5 shows the quantitative analysis of cytokine gene expression levels in drNKs by qRT-PCR, confirming ten cytokines (CCL5, IFN-γ, CXCL11, CXCL12, GDNF, VEGF, XCL1, IL16, LIF, and LTB) that are more highly expressed in drNKs compared to control NK-92 and iPS-NK cells.

FIG. 6 shows the results of identifying the conditioned media of each CD56^{dim}pNK and drNK cell using a human proteome cytokine array. A of FIG. 6 shows the quantitative analysis of 56 types of secreted proteins in the conditioned media of drNK cells. B of FIG. 6 shows the quantitative analysis of 28 types of increased secreted proteins in the conditioned media of drNK cells compared to the conditioned media of CD56^{dim}pNK cells. C of FIG. 6 shows the results of confirming DPP4, M-CSF, and BDNF proteins selectively secreted in the conditioned media of drNK cells.

FIG. 7 shows the results of confirming the damaged neuron-clearing effect of drNK and verifying its dependency on CD16 expression. A of FIG. 7 shows the ROS-positive labeled (MitoSox Red) SH-SY5Y neuronal cell model induced by H₂O₂-mediated damage. B of FIG. 7 shows the results confirming the significantly superior damaged neuron-clearing effect of drNK compared to the control under co-culture conditions of NK cells and ROS-positive damaged neuronal cells. C of FIG. 7 shows a schematic diagram of a cytotoxicity assay using an anti-CD16 antibody. D of FIG. 7 shows the analysis of the correlation between the damaged neuron-clearing effect and CD16 expression, which confirms that the clearing effect is most significantly affected by the anti-CD16 antibody in drNK cells with the highest CD16 expression, thereby confirming that the clearing activity of drNK cells is associated with CD16 expression.

FIG. 8 shows the results confirming the significantly superior effects of SCP/SCP-SCs and drNK cells on the neurite outgrowth of injured neurons compared to the control. A of FIG. 8 is a schematic diagram of a nerve recovery/regeneration assay in axotomy or partial nerve injury models by treatment with the control regeneration factor NGF, SCPs, SCs, NCSCs, and NK cells, either alone or in co-culture. B of FIG. 8 shows the results confirming neurite length recovery/regeneration from drNK, SCP-SC, and drNK+SCP-SC treatments, either alone or in co-culture, in stem cell-derived neurons after neurite transection injury, based on phase-contrast microscopic cell images and TUJ1-positive neuronal marker staining, demonstrating not only the neuro-regenerative effect of drNKs and SCP-SCs, but also the synergistic effect of drNK+SCP-SC. C of FIG. 8 shows the comparative analysis of the neurite injury recovery/regeneration effect of SCP-derived SCs (SCP-SCs) and drNKs of the present invention, administered either alone or in combination in a partially injured neuronal model, compared to that of NGF, CD56^{dim}pNK, and primary cultured Schwann cells (pSCs) as controls. D of FIG. 8 shows the comparative analysis of neurite injury recovery/regeneration between conventional control SCPs (NCSCs) and the SCPs of the present invention, and between conventional control CD56^{dim}pNKs and the drNKs of the present invention.

FIG. 9 shows the results confirming the promotion of sciatic nerve regeneration and therapeutic effects of transplantation of SCPs and NK cells, either alone or in combination, in an animal model. A of FIG. 9 is a schematic diagram of the animal model experiment. B and C of FIG. 9 show the superior effects of SCPs in GFP⁺ SCP infiltration/engraftment and increasing myelination marker MBP-positive myelinating cells, compared to control NCSCs. D of FIG. 9 shows the results of Rotarod test analysis of motor function recovery following nerve regeneration in the animal model of A of FIG. 10, confirming that the group transplanted with SCPs of the present invention exhibited significantly improved motor function compared to the control NCSC group. E of FIG. 9 shows the results that a combined treatment of SCP+drNK exhibits a significantly superior neuro-regenerative effect compared to single treatment.

FIG. 10 shows the neuro-regenerative and therapeutic effects of transplantation of SCP-SCs and NK cells, either alone or in combination, in a partial sciatic nerve injury animal model. A of FIG. 10 shows the results of sampling sciatic nerves from each group four weeks after single or combined transplantation of NGF, CD56^{bright}pNK, drNK, and SCP-SC cells in the partial sciatic nerve injury model, and confirming the therapeutic effect on peripheral neuropathy by hematoxylin and eosin (H&E) staining; it was confirmed that in the single treatment groups, the drNKs of the present invention exhibited superior nerve recovery/regeneration effects compared to conventional CD56^{bright}pNKs, and that the SCP-SC+NK combined treatment group exhibited relatively superior regenerative therapeutic effects compared to the single treatment groups. B and C of FIG. 10 show the results of confirming the therapeutic effect of control drug NGF treatment or NKs and SCP-SCs transplantation, alone or in combination, through quantitative analysis of neuronal marker TUJ1-positive immunohistochemical images; confirming that compared to the control drug NGF, the SCP-SC, CD56^{bright}pNK, and drNK single treatment groups exhibited significantly enhanced nerve regenerative effects, and that the drNKs of the present invention exhibited superior nerve recovery/regeneration effects compared to conventional CD56^{bright}pNKs.

FIG. 11 shows the neuro-regenerative and therapeutic effects of transplantation of SCP-SCs and NK cells, either alone or in combination, in the animal model of FIG. 10. A of FIG. 11 shows the results of immunostaining Schwann cell marker S100 in nerve bundles at the injury site. B of FIG. 11 shows the quantitative analysis of S100 gene expression from total mRNA obtained from nerve bundles at the injury site. C of FIG. 11 shows the results of immunostaining myelin marker MBP in nerve bundles at the injury site. D of FIG. 11 shows the quantitative analysis of MBP gene expression from total mRNA obtained from nerve bundles at the injury site. Immunohistochemical staining of the myelin marker MBP and qRT-PCR analysis confirmed that, compared to SCP-SC or drNK single transplantation groups, the SCP-SC⁺ drNK combined transplantation group exhibited the most remarkable increase in S100- and MBP-positive cells; thereby confirming that myelin sheath regeneration was most enhanced in the SCP-SC+drNK combined transplantation group, contributing to the improvement of nerve functionality.

FIG. 12 shows the neuro-regenerative and therapeutic effects of transplantation of SCP-SCs and NK cells, either alone or in combination, in the animal model of FIG. 10 through behavioral testing (Rotarod). Among the single transplantation groups, the SCP-SC transplantation group exhibited the most superior motor function improvement and the drNK transplantation group exhibited greater motor function improvement compared to the CD56^{bright}pNK group, and among the comparative groups, the drNK+SCP-SC combined transplantation group exhibited the most significant improvement in motor function.

FIG. 13 shows the therapeutic effects of treatment with NK cells and Schwann cells, either alone or in combination, in a Parkinson's disease (PD) cell model. A of FIG. 13 shows the results of inducing differentiation in undifferentiated SH-SY5Y neurons to mature neurons. B of FIG. 13 shows the results of establishing a PD cell model by treating rotenone (10 μM) and verifying the properties of the PD model by confirming a-synuclein expression through immunofluorescence staining. C of FIG. 13 shows the results of analyzing the endocytosis of α-synuclein in NK cells by immunoblot analysis, confirming that drNK cells exhibit higher uptake of α-synuclein compared to NK92 cells. D of FIG. 13 shows the results of evaluating the cytotoxicity of NK cells against the PD cell model, confirming that drNK cells exhibited significantly higher killing activity than NK92, pNK, and iPSC-NK cells (showing superior cytotoxicity at both E:T ratios of 0.25:1 and 0.5:1). E of FIG. 13 shows the results of axonal length analysis performed after treating the PD model with conditioned media derived from NK cells (drNK-CM), conditioned media derived from SCP-derived Schwann cells (SCP-SC-CM), and a 50:50 mixture thereof, in order to evaluate the neurogenerative effect. Neurite outgrowth-promoting effects were observed in both single and mixed conditioned media treatment groups, with the mixed conditioned media treatment group exhibiting the most prominent neurite outgrowth-promoting effect.

FIG. 14 shows the pain-relieving effect in a sciatic nerve injury pain mouse model. A of FIG. 14 shows the results of inducing partial sciatic nerve injury in 8-week-old immunodeficient male Balb/c-nude mice, followed by systemic transplantation at the injury site of SCP-derived Schwann cells (SCP-SCs) or drNK cells, either alone or in combination, and subsequently assessing the degree of nerve regeneration and functional recovery through histological analysis and behavioral evaluation over three weeks. B of FIG. 14 shows that pain sensory recovery was assessed by evaluating the mechanical stimulus response (Von Frey test) using Von Frey filaments, which confirmed the reduction of pain response not only in the drNK and SCP-SC single transplantation groups but also in the combined transplantation group, and the most superior pain-relieving effect was observed in the combined transplantation group.

### DETAILED DESCRIPTION OF PREFERRED EMBODIMENTS

The present invention will be described in detail as follows. Meanwhile, each description and embodiment disclosed herein may also be applied to other descriptions and embodiments. That is, all combinations of various elements disclosed herein fall within the scope of the present invention. Further, the scope of the present invention is not limited by the specific description described below.

To achieve the above objects, one aspect of the present invention provides a pharmaceutical composition for preventing or treating a neurological disease, comprising Schwann cell precursors (SCPs) or Schwann cells (SCs) differentiated therefrom; and natural killer (NK) cells as active ingredients.

Specifically, the present invention provides a pharmaceutical composition for preventing or treating a neurological disease, comprising Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10, and a combination thereof; and natural killer (NK) cells expressing one or more selected from the group consisting of CD56+, CD16+, and a combination thereof.

The present inventors have, for the first time, identified that administration of the composition to peripheral and central nervous system injury models resulted in gene expression characteristics different from those of conventional Schwann cell precursors (NCSCs), a superior secreting ability of the neurotrophic factor GDNF compared to conventional NCSCs, and superior regenerative and therapeutic effects on an injured nervous system.

In particular, the Schwann cell precursors (SCPs) of the present invention exhibited significantly higher expression levels of the neurotrophic factors GDNF and IGFBP-2, and higher secretion levels of proteins affecting nerve regeneration and growth, compared to the conventionally known representative Schwann cell precursor NCSCs.

In addition, the directly reprogrammed natural killer (drNK) cells of the present invention exhibited higher expression of NK cell activation receptors such as CD69, NKG2D, DNAM-1, and NKp46, compared to control NK cells. Ten cytokine/chemokine genes overexpressed in the drNK cells of the present invention were identified, and three proteins were uniquely identified in the drNK cells.

Moreover, when the Schwann cell precursors (SCPs) of the present invention or Schwann cells (SCs) differentiated therefrom, and natural killer (NK) cells were combined and administered to peripheral and central nervous system injury models, it was confirmed that the SCP-SC+NK combined treatment group exhibited superior neurogenic growth and regenerative therapeutic effects compared to the single treatment groups.

In particular, the directly reprogrammed natural killer (drNK) cells of the present invention, compared to control NK cells, when administered to peripheral and central nervous system injury models in combination with SCP-SCs, exhibited relatively superior neurogenic growth and regenerative therapeutic effects in the combined treatment group compared to the single treatment groups.

This indicates that the composition of the present invention not only exhibits higher levels of gene expression or protein secretion that affect nerve regeneration or growth compared to known SCPs or NK cells, but also provides superior neurogenic growth and regenerative effects when used in combination rather than as single treatments, thereby suggesting its utility in the prevention or treatment of neurological diseases.

In one embodiment, the Schwann cell precursors (SCPs) of the present invention may be prepared by a method of preparing SCPs from pluripotent stem cells (PSCs), comprising: (a) culturing pluripotent stem cells in a first medium comprising SB431542 and CT99021; and (b) culturing the cells cultured from (a) in a second medium that further comprises Neuregulin-1 (NRG1), but the method is not limited thereto.

As used herein, the term “SB431542” refers to a specific inhibitor of Transforming Growth Factor-β(TGF-β) which has the structure of Formula 1 below.

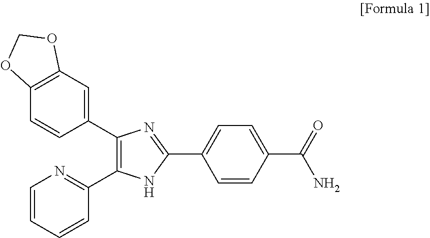 [SMILES: NC(=O)c1ccc(-c2nc(-c3ccc4c(c3)OCO4)c(-c3ccccn3)[nH]2)cc1]

Specifically, SB431542 may be comprised at a concentration of 1 μM to 100 μM, more specifically 1 μM to 50 μM, still more specifically 1 μM to 30 μM, and even more specifically 5 μM to 25 μM, but is not limited thereto.

As used herein, the term “CT99021” refers to CHIR-99021 (CT99021), a GSK-3α/β inhibitor, and may also be referred to as CT99021, CHIR99021, CHIR 99021,CHIR-99021, or CT-99021. CT99021 has the structure of Formula 2 below.

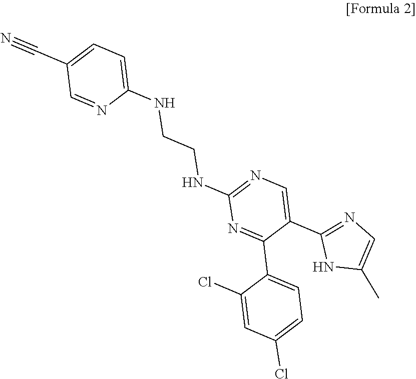 [SMILES: Cc1cnc(-c2cnc(NCCNc3ccc(C#N)cn3)nc2-c2ccc(Cl)cc2Cl)[nH]1]

Specifically, CT99021 may be comprised at a concentration of 1 μM to 100 μM, more specifically 1 μM to 50 μM, still more specifically 1 μM to 10 μM, and even more specifically 1 μM to 5 μM, but is not limited thereto.

As used herein, “Neuregulin-1 (NRG1)” refers to a protein encoded by the NRG1 gene, which acts on the EGFR receptor. Specifically, NRG1 may be comprised at a concentration of 1 ng/ml to 1000 ng/ml, more specifically 10 ng/ml to 500 ng/ml, still more specifically 20 ng/ml to 200 ng/ml, and even more specifically 30 ng/ml to 100 ng/ml, but is not limited thereto.

In another embodiment, the first medium of (a) may further comprise FGF2, and the second medium of (b) may further comprise Stem Regenin I (SR I), but are not limited thereto.

As used herein, “Fibroblast Growth Factor 2 (FGF2)” is a fibroblast growth factor, which may be used interchangeably with the term “basic fibroblast growth factor (bFGF)” or “FGF-β”. Specifically, FGF2 may be comprised at a concentration of 1μg/ml to 100 μg/ml, more specifically 1 μg/ml to 50 μg/ml, still more specifically 5 μg/ml to 50 μg/ml, and even more specifically 10 μg/ml to 30 μg/ml, but is not limited thereto.

As used herein, “StemRegenin I (SR I)” is an aryl hydrocarbon receptor inhibitor and refers to 4-(2-((2-(Benzo[b]thiophen-3-yl)-9-isopropyl-9H-purin-6-yl)amino)ethyl) phenol hydrochloride. StemRegenin I may be further comprised in the second medium for preparing Schwann cell precursors (SCPs). SR I may be comprised at a concentration of 1 μM to 100 μM, specifically 1 μM to 50 μM, more specifically 1 μM to 10 μM, and even more specifically 1 μM to 5 μM, but is not limited thereto.

Another specific aspect of the present invention provides Schwann cell precursors (SCPs) prepared by the above method. The Schwann cell precursors prepared by the method of the present invention are Schwann cell precursors differentiated from pluripotent stem cells (PSCs), and have multipotency to differentiate into Schwann cells, melanocytes, etc., a high proliferation rate (expandability), and long-term maintainability. In addition, the Schwann cell precursors may express one or more specific marker genes of Schwann cell precursors selected from the group consisting of GAP43, SOX10, IGFBP2, and a combination thereof, but are not limited thereto.

As used herein, the term “Schwann cell precursor (SCP)” refers to an intermediate stage that Schwann cells undergo during the neural crest developmental process, specifically, an intermediate cell stage between neural crest (stem) cells (NC(S)Cs) and immature, pre-myelinating Schwann cells. The Schwann cell precursors can differentiate into Schwann cells.

As used herein, the term “pluripotent stem cell (PSC)” refers to an undifferentiated stem cell having the ability to differentiate into cells of all three germ layers (endoderm, mesoderm, and ectoderm). Under ex vivo culture conditions, undifferentiated pluripotent stem cells have multipotency and the ability to self-renew (self-replicate) while retaining normal karyotypes. In the present invention, multipotency may include both pluripotency and multipotency. Pluripotent stem cells may include embryonal carcinoma (EC) cells, embryonic stem(ES) cells, and embryonic germ (EG) cells. Specifically, the pluripotent stem cells of the present invention may be human embryonic stem cells (hESCs) or induced pluripotent stem cells (hiPSCs); however, any origin is included without limitation as long as the cells possess pluripotency.

In one embodiment of the present invention, upon reviewing the differences between the SCPs of the present invention and neural crest stem cells (NCSCs), which are conventionally known representative Schwann cell precursors, it was confirmed that the expression levels of the neurotrophic factors GDNF and IGFBP-2 were significantly higher in the SCPs than in the NCSCs, and that the secretion levels of proteins affecting nerve regeneration and growth were also higher. Accordingly, it is evident that the SCPs of the present invention can exhibit greater effects on nerve growth and regeneration compared to conventional NCSCs by virtue of the neurotrophic factors secreted at higher levels.

In another embodiment, Schwann cells derived from human pluripotent stem cell-Schwann cells (PSC-SCP-SCs) of the present invention may be prepared by a method of preparing SCs from PSCs, comprising: (a) culturing pluripotent stem cells in a first medium comprising SB431542 and CT99021; (b) culturing the cells cultured in (a) in a second medium that further comprises Neuregulin-1 (NRG1); (c) recovering SCPs from the cultured medium; and (d) culturing the recovered SCPs in a third medium comprising FBS and NRG1.

In another embodiment, the third medium of (d) may further comprise one or more selected from the group consisting of retinoic acid, forskolin, and PDGF-BB, but is not limited thereto.

As used herein, the term “retinoic acid” refers to a metabolic product generated when vitamin A is degraded in the body and has the chemical formula C₂₀H₂₈O₂. Retinoic acid is known to have effects such as inhibition of colorectal cancer and treatment of rheumatism. Specifically, the retinoic acid may be included in a medium at a concentration of 1 nM to 300 nM, more specifically 10 nM to 200 nM, and still more specifically 50 nM to 150 nM, but is not limited thereto.

As used herein, the term “forskolin” refers to a labdane diterpene produced from the Indian Coleus plant (Plectranthus barbatus). Specifically, the forskolin may be included in a medium at a concentration of 1 μM to 100 μM, more specifically 1 μM to 50 M, and still more specifically 1 μM to 10 μM, but is not limited thereto.

As used herein, the term “platelet-derived growth factor-BB (PDGF-BB)” refers to a homodimer of PDGFB encoded by the PDGFB gene. Specifically, the PDGF-BB may be included in a medium at a concentration of 1 ng/ml to 100 ng/ml, more specifically 1 ng/ml to 50 ng/ml, and still more specifically 5 ng/ml to 15 ng/ml, but is not limited thereto.

As used herein, the term “Schwann cell (SC)” refers to a glial cell in the peripheral nervous system, which plays roles in myelin sheath formation, nerve impulse transmission, and secretion of neurotrophic factors, and is particularly known to affect neuronal survival and axonal growth.

Another aspect of the present invention provides Schwann cells prepared by the above method. The Schwann cells prepared by the method of the present invention are Schwann cells prepared from Schwann cell precursors derived from pluripotent stem cells, and exhibit positive expression for Schwann cell-specific marker genes such as S100B and SOX10.

In another embodiment, the natural killer (NK) cell may express one or more selected from the group consisting of CD56⁺, CD16⁺, and a combination thereof, but is not limited thereto. Specifically, the natural killer (NK) cell may express one or more selected from the group consisting of CD56^{dim}, CD56^{bright}, CD56^{superbright}, CD16^{dim}, CD16^{bright}, CD16^{superbright}, and a combination thereof, but is not limited thereto.

In one example, the natural killer (NK) cell may be: (1) pNK isolated from human peripheral blood; (2) CD56^{dim}CD16^{bright} pNK isolated from human peripheral blood; (3) CD56^{bright}CD16^{bright} pNK obtained by activating pNK isolated from human peripheral blood with IL-2 or IL-15 cytokine; and (4) an immortalized CD56^{bright}CD16^{dim} NK cell line (NK92), but is not limited thereto.

In another embodiment, the directly reprogrammed natural killer (drNK) cells may be prepared by a method of preparing drNK cells from isolated cells, the method comprising: (a) introducing a reprogramming factor into the isolated cells; and (b) from the day after introducing the reprogramming factor, culturing the cells in (a) i) in a first medium comprising cytokines, growth factors, and glycogen synthase kinase 3β (GSK3β) inhibitors to increase the efficiency of direct reprogramming, and ii) in a second medium comprising cytokines, growth factors, and aryl hydrocarbon receptor (AHR) inhibitors to promote the production of natural killer cells.

Another specific aspect of the present invention provides directly reprogrammed natural killer (drNK) cells prepared by the above method. The directly reprogrammed natural killer cells produced by the method of the present invention may express one or more selected from the group consisting of CD56^{superbright}, CD16^{superbright}, and a combination thereof, but are not limited thereto.

In another embodiment, the directly reprogrammed natural killer (drNK) cells may overexpress one or more genes selected from the group consisting of CCL5, IFN-Y, CXCL11, CXCL12, GDNF, VEGF, XCL1, IL16, LIF, and LTB, compared to a control group, but are not limited thereto.

In another embodiment, the directly reprogrammed natural killer (drNK) cells may express one or more proteins selected from the group consisting of DPP4, M-CSF, and BDNF, but are not limited thereto.

As used herein, the term “cytokines” refers to various proteins of relatively small size that are produced in cells and used for cell signaling, and can affect other cells, including themselves. They are generally known to be involved in the immune response to inflammation or infection, but are not limited thereto. Specifically, the cytokines may be IL-2, IL-3, IL-5, IL-6, IL-7, IL-11, IL-15, BMP4, Activin A, Notch ligand, G-CSF, or SDF-1, but are not limited thereto.

As used herein, the term “growth factor” means a polypeptide that promotes the division, growth, and differentiation of various cells, and includes epidermal growth factor (EGF), platelet-derived growth factor-AA (PDGF-AA), insulin-like growth factor 1 (IGF-1), transforming growth factor-β (TGF-β), or fibroblast growth factors (FGFs), but is not limited thereto.

For the purpose of the present invention, the cytokines and growth factors are included in the medium for directly reprogramming isolated cells into lineage-converted cells, and the types of cytokines and growth factors are not limited as long as they are used for direct reprogramming.

As used herein, the term “natural killer (NK) cell” refers to a key innate immune cell that immediately recognizes and eliminates infections caused by viruses, germs (bacteria), fungi, and parasites, as well as abnormal autologous cells. Unlike T cells that recognize target cells by expressing antigen-specific receptors, NK cells recognize, without antigen specificity or human leukocyte antigen (HLA) matching, abnormal changes in target cells such as the balance of inhibitory or activating receptors (e.g., killer immunoglobulin receptors (KIR), natural cytotoxicity receptors (NCR), DNAX accessory molecule-1 (DNAM-1), and NK group 2 member D (NKG2D)); loss of surface major histocompatibility complex (MHC) class I antigens; and accumulation of abnormal proteins, and exert contact-dependent cytotoxicity through various mechanisms. Unlike T cells that can cause graft-versus-host disease (GVHD) against non-self allogeneic cells with mismatched human leukocyte antigens (HLAs), allogeneic NK cells have been confirmed to exhibit minimal side effects of GVHD and, rather, to display strong therapeutic effects.

As used herein, the term “direct reprogramming” refers to a method of converting the lineage of a specific cell into a target cell having completely different characteristics by controlling the global gene expression pattern, etc. thereof. The direct reprogramming may be a concept that includes reprogramming, differentiation, direct-differentiation, dedifferentiation, direct-dedifferentiation, conversion, direct-conversion, trans-differentiation, or direct trans-differentiation of cells, but is not limited thereto.

The direct reprogramming may refer to performing “cell transformation” by introducing an oligonucleotide or a vector containing a foreign gene or DNA into a cell, or refer to the change of a cell to a different state. The term “differentiation” refers to a phenomenon in which daughter cells produced by cell division acquire a function different from that of the original parent cell, and as used herein, the term “direct reprogramming” may be used interchangeably with “induction of direct cell transformation”, “direct cell transformation”, or “cell transformation”.

For the purpose of the present invention, a natural killer cell refers to that obtained through direct reprogramming, and may be used interchangeably with “directly reprogrammed natural killer (drNK) cell”.

As used herein, the term “isolated cells” is not particularly limited, but may be cells whose lineage has already been specified, for example, germ cells, somatic cells, or progenitor cells. The “somatic cells” refer to all cells in which differentiation constituting animals and plants has been completed except for germ cells. The “progenitor cell” refers to a mother cell which does not express a differentiated trait, but has a differentiation fate, when it has been identified that a cell corresponding to its progeny expresses a certain differentiation trait. For example, hematopoietic stem cells correspond to progenitor cells of blood cells, and mesenchymal stem cells correspond to progenitor cells of mesenchymal cells.

The isolated cells may be cells derived from a human, but are not limited thereto, and cells derived from various individuals may also fall within the scope of the present invention. In addition, the isolated cells of the present invention may include both in vivo or ex vivo cells.

In one example, the isolated cells may be somatic cells, and in another example, the isolated cells may be somatic cells excluding NK cells, and in still another example, the isolated cells may be one or more selected from the group consisting of blood cells and fibroblasts, but are not limited thereto. For example, the blood cells may be peripheral blood mononuclear cells (PBMC), but are not limited thereto.

As used herein, the term “direct reprogramming-inducing factor” refers to a gene (or polynucleotide) that can be introduced into a cell to induce cell transformation, or a protein encoded thereby. The direct reprogramming-inducing factor may vary depending on the target cell to be obtained through reprogramming, and the type of cell before cell transformation. The cell transformation using the direct reprogramming-inducing factor refers to the induction of conversion to a target cell by controlling the entire gene expression pattern of the cell, and the cell may be induced to convert into a target cell having the gene expression pattern of a desired cell type by introducing the direct reprogramming-inducing factor into the cell and culturing the same for a certain period of time. As used herein, the “direct reprogramming-inducing factor” may be used interchangeably with “direct cell transformation-inducing factor”, “cell transformation-inducing factor”, or “reprogramming-inducing factor”.

As used herein, the “introduction of a direct reprogramming-inducing factor” may be: a method of administering a direct reprogramming-inducing factor to a cell culture medium; a method of directly injecting a direct reprogramming-inducing factor into cells; a method of increasing or decreasing the expression level of a direct reprogramming-inducing factor present in a cell; a method of transforming a cell with an expression vector comprising a gene encoding a direct reprogramming-inducing factor; a method of modifying a gene sequence to increase or decrease the expression of a gene encoding a direct reprogramming-inducing factor; a method of introducing an exogenously expressed gene encoding a direct reprogramming-inducing factor; a method of treating a substance having an effect of inducing or suppressing the expression of a direct reprogramming-inducing factor; and a method of increasing or decreasing the expression level of a direct reprogramming-inducing factor in a cell through a combination thereof; but the method is not limited thereto as long as it can increase or decrease the expression level of a direct reprogramming-inducing factor. In particular, the introduction of a direct reprogramming-inducing factor may be inducing expression of a direct reprogramming-inducing factor under desired times and conditions. Specifically, the method of introducing a direct reprogramming-inducing factor into a cell may be a method of administering a direct reprogramming-inducing factor to a cell culture medium or a method of transforming a cell with an expression vector comprising a gene encoding a direct reprogramming-inducing factor, but is not limited thereto.

In one example, the method of directly injecting a direct reprogramming-inducing factor into a cell may be performed by selecting any method known in the art, but is not limited thereto, and may be performed by appropriately selecting from the methods using microinjection, electroporation, particle bombardment, direct muscle injection, an insulator, and a transposon.

In one embodiment of the present invention, fresh primary NK cells isolated from PBMCs (pNK), pNK cells activated with IL-2 and IL-15 (ApNK), or an NK cell line (NK92, ATCC) were used as controls to compare the drNKs of the present invention with conventional NK cells. It was confirmed that according to the expression of CD56 and CD16 markers, the major cell populations were distinguished into the following cellular phenotypes: drNK: CD56^{superbright}CD16^{superbright}, NK92: CD56^{bright}CD16^{dim}, pNK (CD56^{dim}pNK): CD56^{dim}CD16^{bright}, ApNK (CD56^{bright}pNK): CD56^{bright}CD16^{bright}. In particular, comparison of NK cell receptor expressions confirmed that the expressions of NK cell activating receptors such as CD69, NKG2D, DNAM-1, and NKp46 were higher in drNKs than in CD56^{dim}pNK.

In another embodiment of the present invention, comparison of the gene expressions of cytokines/chemokines in the drNKs of the present invention with those in control NK cells revealed ten overexpressed cytokines/chemokines (i.e., CCL5, IFN-Υ, CXCL11, CXCL12, GDNF, VEGF, XCL1, IL16, LIF, and LTB), and identified three proteins uniquely expressed in drNK (i.e., DPP4, M-CSF, and BDNF).

In another embodiment of the present invention, upon examining the correlation between the damaged neuron-clearing effect of natural killer cells and the expression level of CD16, it was confirmed that the damaged neuron-clearing effect was reduced by the treatment of anti-CD16 antibody to 58.1% in CD56^{dim}pNK, 50.1% CD56^{bright}pNK, 82.6% in NK92, and 30.9% in drNK. Consequently, it was confirmed that the inhibitory effect of the anti-CD16 antibody is proportional to the expression level of CD16, and in the drNK cells of the present invention, the anti-CD16 antibody exhibited the greatest inhibitory effect of 69.1%.

As used herein, the term “neurological disease” refers to a disease related to the nervous system, which may be caused by damage, degeneration, or loss of function of formed myelin (sheath) or axons due to external or internal factors, or by the loss or damage of neurons.

In the present invention, the neurological disease specifically may be brain tumor, cerebral infarction, hypertensive cerebral hemorrhage, cerebral contusion, cerebral arteriovenous malformation, brain abscess, encephalitis, hydrocephalus, epilepsy, concussion, cerebral palsy, mild cognitive impairment, dementia, spinal cord tumor, spinal arteriovenous malformation, spinal cord infarction, pain, headache, migraine, Alzheimer's disease, Parkinson's disease, Huntington's disease, amyotrophic lateral sclerosis (ALS), Batten disease, Kearns-Sayre syndrome (KSS), chronic progressive external ophthalmoplegia (CPEO), mitochondrial encephalomyopathy, lactic acidosis, and stroke-like episodes (MELAS syndrome), myoclonic epilepsy with ragged-red fibers (MERRF) syndrome, neurogenic weakness with ataxia and retinitis pigmentosa (NARP) syndrome, Leigh syndrome, mitochondrial recessive ataxia syndrome (MIRAS), degenerative neurological diseases, schizophrenia, schizophreniform disorder, attention deficit hyperactivity disorder (ADHD), personality disorders, autism, post-traumatic stress disorder (PTSD), anxiety disorders, panic disorders, depression, chronic stress-related depression, delusional disorders, obsessive-compulsive disorders, anorexia nervosa, bulimia nervosa, obesity, cerebral ischemic diseases, neurodegenerative diseases, diabetic neuropathy, traumatic nerve injury, neurodegenerative disorders, neuropathic pain, epilepsy, chronic neuropathic pain, Guillain-Barré syndrome, myasthenia gravis, Rett syndrome, central sleep apnea, peripheral neuropathy, Charcot-Marie-Tooth disease, spinal muscular atrophy (SMA), autoimmune encephalitis, chronic traumatic encephalopathy (CTE), myotonic dystrophy, multiple sclerosis, schwannoma, neurofibromatosis, chronic inflammatory demyelinating polyneuropathy (CIDP), polyneuropathy, and neurinoma; but is not limited thereto.

As used herein, the term “prevention” refers to all acts of suppressing or delaying the onset of a neurological disease by using a pharmaceutical composition for preventing or treating a neurological disease, comprising the Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10, and a combination thereof; and natural killer (NK) cells expressing one or more selected from the group consisting of CD56⁺, CD16⁺, and a combination thereof.

As used herein, the term “treatment” refers to all acts of alleviating or curing the symptoms of a neurological disease by using a pharmaceutical composition for preventing or treating a neurological disease, comprising the Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10, and a combination thereof; and natural killer (NK) cells expressing one or more selected from the group consisting of CD56⁺, CD16⁺, and a combination thereof.

As used herein, the term “pharmaceutical composition” may comprise a pharmaceutically acceptable carrier, and may be formulated according to a conventional method into oral dosage forms such as powders, granules, tablets, capsules, suspensions, emulsions, syrups, and aerosols, as well as topical agents, suppositories, and sterile injectable solutions.

The pharmaceutically acceptable carrier may include those conventionally used in the art, such as lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and minerals, but is not limited thereto. Further, the pharmaceutical composition of the present invention may comprise diluents or excipients such as fillers, extenders, binders, wetting agents, disintegrants, and surfactants, and other pharmaceutically acceptable additives.

The pharmaceutical composition of the present invention may be formulated as a solid preparation for oral administration, including a tablet, a pill, a powder, a granule, a capsule, etc. Such solid preparation may comprise at least one excipient, for example, starch, calcium carbonate, sucrose or lactose, gelatin, etc., and lubricants such as magnesium stearate or talc, but is not limited thereto.

The pharmaceutical composition of the present invention may be formulated as a liquid preparation for oral administration, including a suspension, an oral liquid, an emulsion, a syrup, etc., and may comprise diluents such as water or liquid paraffin, wetting agents, flavoring agents, aromatics, preservatives, etc., but is not limited thereto.

The pharmaceutical composition of the present invention may be formulated for parenteral administration, including a sterile aqueous solution, a non-aqueous solvent, a suspension, an emulsion, a lyophilized preparation, and a suppository. The non-aqueous solution and the suspension may include propylene glycol, polyethylene glycol, vegetable oil such as olive oil, injectable ester such as ethyloleate, etc, but are not limited thereto. The base for suppositories may include witepsol, macrogol, tween 61, cacao butter, laurin butter, glycerogelatin, etc., but is not limited thereto.

In the present invention, the dosage of the pharmaceutical composition varies depending on the condition, body weight, age, and severity of the disease of the patient, as well as the dosage form, administration route, and administration period; but may be appropriately selected by those skilled in the art.

The pharmaceutical composition of the present invention may be administered to mammals such as rats, mice, livestock, and humans through various routes, for example, oral, intraperitoneal, intravenous, intramuscular, subcutaneous, intrauterine, intradural, or intracerebrovascular injection.

In one embodiment of the present invention, upon examination of neurite outgrowth induced by the SCP/SCP-SC and NK cells of the present invention, it was found that the SCP-SC single-treatment group and the SCP-SC and drNK combined-treatment group exhibited remarkably greater neurite outgrowth compared to the control group; and in particular, the SCP-SC and drNK combined-treatment group showed significantly greater neurite outgrowth than the SCP-SC single-treatment group. In addition, in the partial nerve injury model, SCP showed higher neurogenic growth effects compared to NCSC, and the SCP and drNK combined-treatment group also exhibited significantly higher neurogenic growth effects compared to the SCP single-treatment group.

In one embodiment of the present invention, transplanting the composition of the present invention into a sciatic nerve injury mouse model showed that the group transplanted with a combination of SCP and drNK exhibited superior recovery of average motor function and superior regenerative therapeutic effects compared to the SCP single-treatment group.

This suggests that administration of induced Schwann cell precursors (SCPs) prepared from the pluripotent stem cells (PSCs) of the present invention and Schwann cells (SCs) differentiated therefrom, each alone or in combination with natural killer cells, to a nerve injury disease model exhibits superior regeneration and therapeutic effects on the damaged nervous system.

Another aspect of the present invention to achieve the above objects provides a cell therapy agent for preventing or treating a neurological disease, comprising Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10,and a combination thereof; and natural killer (NK) cells expressing one or more selected from the group consisting of CD56⁺, CD16⁺, and a combination thereof.

The terms used herein are as described above.

As used herein, the term “cell therapy agent” refers to a pharmaceutical product for treatment, diagnosis, and prevention (U.S. FDA guidance) containing cells or tissues prepared from individuals via isolation, culture, and specialized manipulations, and to a pharmaceutical product for treatment, diagnosis, and prevention used in a series of steps such as proliferating and selecting autologous, homologous, or heterologous living cells ex vivo, or modifying the biological characteristics of cells through other methods, so as to restore the function of cells or tissues.

The cell therapy agent composition may have efficacy in preventing or treating a neurological disease by comprising Schwann cell precursors produced by the method of the present invention, Schwann cells differentiated therefrom, and directly reprogrammed natural killer cells.

The cell therapy agent composition of the present invention may contain the Schwann cell precursors, Schwann cells, and natural killer cells at 1.0×10⁵ cells/mL to 1.0×10¹⁰ cells/mL, specifically 1.0×10⁶ cells/mL to 1.0×10⁹ cells/mL based on the total weight of the composition, but is not limited thereto.

The cell therapy agent composition may be administered by formulating it into a pharmaceutical formulation in the form of unit dosage suitable for administration to the body of a patient by conventional methods in the pharmaceutical field, and contains an effective amount by a single dose or in divided doses. For this purpose, a formulation for parenteral administration may preferably include injection formulation such as an injection ampoule, infusion formulation such as an infusion bag, and spray formulation such as an aerosol, etc. The injection ampoule may be mixed with an injection solution such as saline solution, glucose, mannitol, and Ringer's solution immediately before use. Further, infusion bags made of polyvinyl chloride or polyethylene may be used, and examples thereof may include infusion bags manufactured by Baxter, Becton Dickinson, Medcep, National Hospital Products, or Terumo.

The pharmaceutical formulation may include one or more conventional pharmaceutically acceptable inactive carriers in addition to the active ingredient, for example, a preservative, analgesic controller, solubilizer, or stabilizer for injection formulation, and a base, excipient, lubricant, or preservative for topical formulation.

The thus-prepared cell therapy agent composition of the present invention or a pharmaceutical formulation thereof may be administered in accordance with any conventional method in the art together with other cells used for treatment of a neurological disease, or in the form of a mixture therewith. Specifically, direct engraftment or transplantation to the diseased area of a patient in need of treatment, or direct transplantation or injection into the abdominal cavity is possible, but the method is not limited thereto. Further, both non-surgical administration using a catheter and surgical administration such as injection or transplantation after incision of the diseased area are possible. In I addition, the composition may also be administered parenterally by a conventional method, for example, transplantation of cells into the hematopoietic system, in addition to direct administration to the lesion.

The cell therapy agent composition of the present invention may be administered in a dose ranging from about 0.0001 mg/kg to 1000 mg/kg, specifically 0.01 mg/kg to 100 mg/kg, once per day or in several divided doses per day. However, it should be understood that the amount of the active ingredient actually administered ought to be determined in light of various relevant factors including the disease to be treated, the severity of the disease, the route of administration, and the body weight, age, and sex of a patient. Therefore, the above dose is not intended to limit the scope of the present invention in any way.

Another aspect of the present invention to achieve the above objects provides a method for preventing or treating a neurological disease, comprising administering to a non-human subject suspected of having a neurological disease, a pharmaceutical composition for preventing or treating a neurological disease that comprises: Schwann cell precursors (SCPs) or Schwann cells (SCs) differentiated therefrom; and directly reprogrammed natural killer (drNK) cells, as active ingredients.

Specifically, a method is provided for preventing or treating a neurological disease, comprising administering to a non-human subject suspected of having a neurological disease, a pharmaceutical composition for preventing or treating a neurological disease comprising Schwann cell precursors (SCPs) expressing one or more selected from the group consisting of GAP43, SOX10, IGFBP2, and a combination thereof, or Schwann cells (SCs) expressing one or more selected from the group consisting of S100B, SOX10, and a combination thereof; and natural killer (NK) cells expressing one or more selected from the group consisting of CD56⁺, CD16⁺, and a combination thereof.

The terms used herein are as described above.

As used herein, the term “administration” means introducing the composition of the present invention into a subject by any suitable method. The administration route of the composition may be administration through any general route as long as it can reach the target tissue, including intraperitoneal administration, intravenous administration, intramuscular administration, subcutaneous administration, intradermal administration, oral administration, topical administration, and intranasal administration, but is not limited thereto.

As used herein, the term “subject” refers to any animal excluding humans having a neurological disease or at risk of having a neurological disease, including monkeys, cattle, horses, sheep, pigs, chickens, turkeys, quails, cats, dogs, mice, rats, rabbits, or guinea pigs. As long as the disease can be effectively prevented or treated by administering the pharmaceutical composition of the present invention into a subject, any type of subject may be included without limitation.

### Mode for Carrying Out the Invention

Hereinafter, the present invention will be described in more detail by way of exemplary embodiments. However, the following embodiments are only exemplary embodiments for illustrating the present invention, and thus the scope of the present invention is not intended to be limited by these examples.

### Example 1: Differentiation of Schwann Cell Precursors (SCPs) from Human Pluripotent Stem Cells (PSCs)

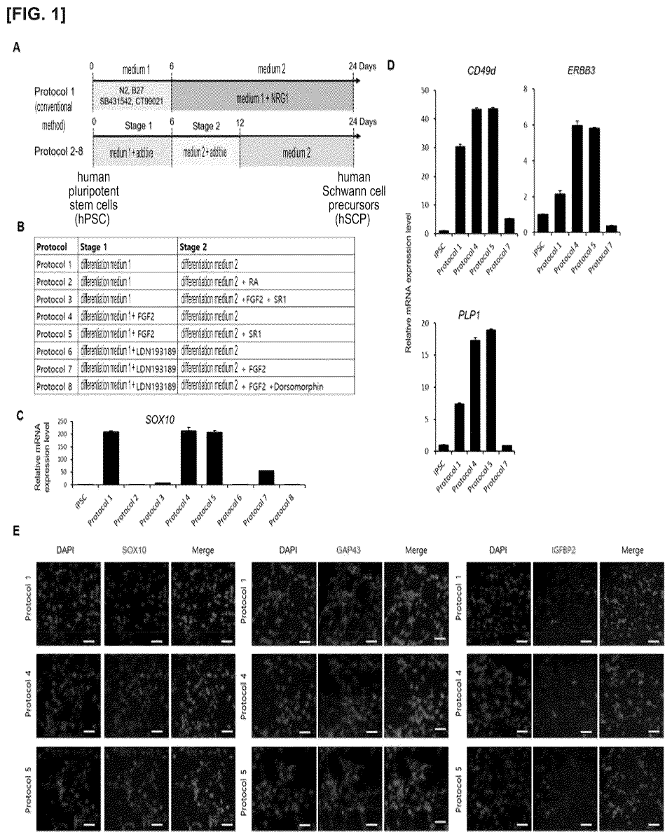

A novel method improved from the conventional production method (Protocol 1 of FIG. 1A) for differentiating pluripotent stem cells into Schwann cell precursors was devised. To evaluate the effects of FGF2, LDN193189, all-trans-retinoic acid (RA), StemRegenin1 (SR1), and dorsomorphin as candidate factors for promoting the differentiation of novel Schwann cell precursors, the characteristics of cells differentiated by eight different methods (Protocols 1 to 8) were comparatively analyzed as shown in A and B of FIG. 1.

First, human pluripotent stem cells (PSCs), such as human induced pluripotent stem cells derived from newborn foreskin fibroblasts (catalog number CRL-2097; ATCC) and human embryonic stem cells (H9 ESC, WiCell), were cultured as follows. For feeder-free culture, the cells were grown in mTeSR1 medium (StemCell Technologies) on dishes coated with growth factor-reduced Matrigel (BD Biosciences), with daily medium changes. To obtain Schwann cell precursors (SCPs) derived from human PSCs, colonized PSCs were replated onto growth factor-reduced Matrigel-coated culture dishes. On the following day, the culture medium was replaced from a human PSC culture medium to a modified neural differentiation medium (NDM) comprising SB431542 and CT99021 and thereby neutralized. The cells were cultured for 6 days to form neural rosettes. Specifically, the NDM comprised advanced DMEM/F12 and Neurobasal medium (1:1 mixture) comprising 1×N2, 1×B27,0.005% BSA, 2 mM Glutamax, 0.11 mM β-mercaptoethanol, 3 μM CT99021 (Tocris Biosciences), and 20 μM SB431542 (Tocris Biosciences), or, as shown in A and B of FIG. 1, further comprised 20 μg/ml FGF2 (Peprotech) or 100 nM LDN193189 (Medchemexpress) in NDM medium. After 6 days of differentiation, the NDM was replaced with a neural induction medium comprising 50 ng/ml NRG1 [Schwann cell precursor differentiation medium (SCPDM)], wherein the SCPDM further comprised, depending on the experimental conditions shown in B of FIG. 1, 100 nM RA (all-trans retinoic acid, Sigma), 20 μg/ml FGF2, 2 μM SR1 (StemRegenin1, Cellagen), or 2 μM dorsomorphin (Medchemexpress). The SCPDM was replaced every two days, and when the cells reached 80% confluence, they were detached with Accutase and further cultured for 6 days. In addition, the cells were proliferated by further culture in SCPDM. After approximately 18 days of differentiation, to confirm the generation of SCPs, the expression of the SCP marker gene SOX10 was analyzed by qPCR, and it was found that SOX10 was highly expressed in Protocols 1, 4, 5, and 7 (C of FIG. 1). Further, the expression levels of CD49d, ERBB3, and PLP1, also known as SCP marker genes, were analyzed by qPCR, and it was confirmed that the expression levels were higher in Protocols 4 and 5, novel differentiation methods, than in Protocol 1, the conventional differentiation method (D of FIG. 1). In particular, ERBB3 expression was observed to be more than 2.7-fold higher in Protocol 4 (average 5.99-fold relative to GAPDH) and Protocol 5 (average 5.81-fold relative to GAPDH) compared to Protocol 1 (average 2.15-fold relative to GAPDH). Likewise, PLP1 expression was observed to be more than 2.3-fold higher in Protocol 4 (average 17.2-fold relative to GAPDH) and Protocol 5 (average 18.9-fold relative to GAPDH) compared to Protocol 1 (average 7.39-fold relative to GAPDH). The expression of Schwann cell precursor marker proteins SOX10, GAP43, and IGFBP2 was confirmed in Protocols 1, 4, and 5 by immunocytochemistry (E of FIG. 1). SCPDM was used for the induction and maintenance of PSC-derived SCPs.

### Example 2: Differentiation of Schwann Cells (SCs) from Schwann Cell Precursors (SCPs)

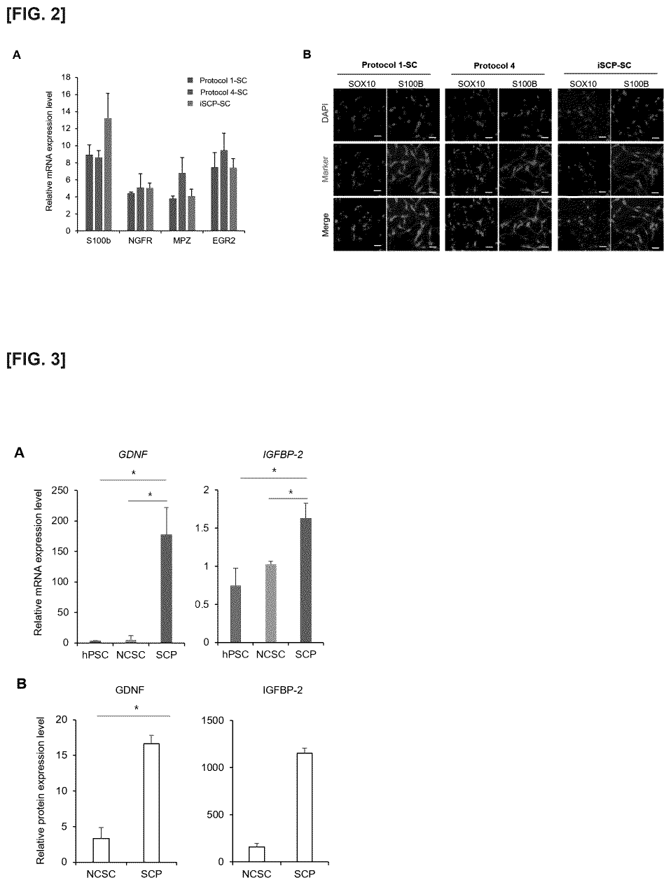

In order to confirm the Schwann cell differentiating-ability of SCPs differentiated by the novel protocol, Schwann cell differentiation was performed together with conventional iSCPs using the previously known SCP-SC differentiation method. To differentiate SCPs into Schwann cells, the above SCPs and SCPs produced by a somatic cell reprogramming method (iSCPs) were cultured on Matrigel-coated plates in Schwann cell differentiation medium (SCDM). The SCDM comprised DMEM comprising 1% FBS, 200 ng/ml NRG1, 4 μM forskolin (Sigma), 100 nM all-trans retinoic acid (RA, Sigma), and 10 ng/ml PDGF-BB. After 3 days of culture, the medium was replaced with SCDM comprising 1% FBS, 200 ng/ml NRG1,and 10 ng/ml PDGF-BB (Thermo Fisher Scientific), but not comprising forskolin or retinoic acid. After another 2 days, the medium was replaced with Schwann cell medium (SCM), which comprised 1% FBS and 200 ng/ml NRG1, but did not comprise forskolin, retinoic acid, or PDGF-BB. The cultured cells were maintained in SCM for expansion. Schwann cells were generated after 2 to 3 days of culture in SCM. On day 7 of differentiation, qPCR analysis of expression levels of SC marker genes S100b, NGFR, MPZ, and EGR2 demonstrated that the expression levels were not significantly different from those of iSCP-SCs with confirmed Schwann cell functions (A of FIG. 2). Furthermore, immunocytochemical analysis confirmed that most of the differentiated SCs were positive for SC lineage-specific proteins S100B and SOX10 (B of FIG. 2). Therefore, it was confirmed that Schwann cell precursors differentiated by the novel method successfully produced Schwann cells in a short period of time (about 7 days), similar to cells differentiated by the conventional method.

### Example 3: Confirmation of Increased mRNA Expression and Protein Secretion of Neurotrophic Factors GDNF and IGFBP-2 in Induced SCPs Compared to Conventional Schwann Cell Precursors (NCSCs)

To confirm the difference in protein-secreting ability between the induced SCP of the present invention and NCSC, a conventionally known representative Schwann cell precursor, NCSC was first differentiated from human PSC (H9 ESC) by the following method.

Specifically, the separated PSC was plated onto a Matrigel-coated culture dish, and on the following day, the culture medium was replaced with an NCSC induction medium (NCSCIM) comprising 1% Probumin (Millipore), 1% penicillin-streptomycin, 1% L-alanyl-L-glutamine (Cellgro), 1% MEM non-essential amino acids, 0.1% Trace elements A (Cellgro), 0.1% Trace elements B (Cellgro), 0.1% Trace elements C (Cellgro), 0.11 mM β-mercaptoethanol, 10 μg/ml transferrin, 50 μg/ml (+)-sodium I-ascorbate (Sigma), 10 ng/ml NRG1 (Peprotech), 200 ng/ml LONG R3 IGF-I (Sigma), 3 μM BIO (Tocris Biosciences), 20 μM SB431542 (Tocris Biosciences), and 8 ng/ml FGF2 (Peprotech). The culture medium was replaced daily. NCSCs were produced after about 20 days of growth in NCSCIM. Unless otherwise specified, all reagents were purchased from Thermo Fisher Scientific.

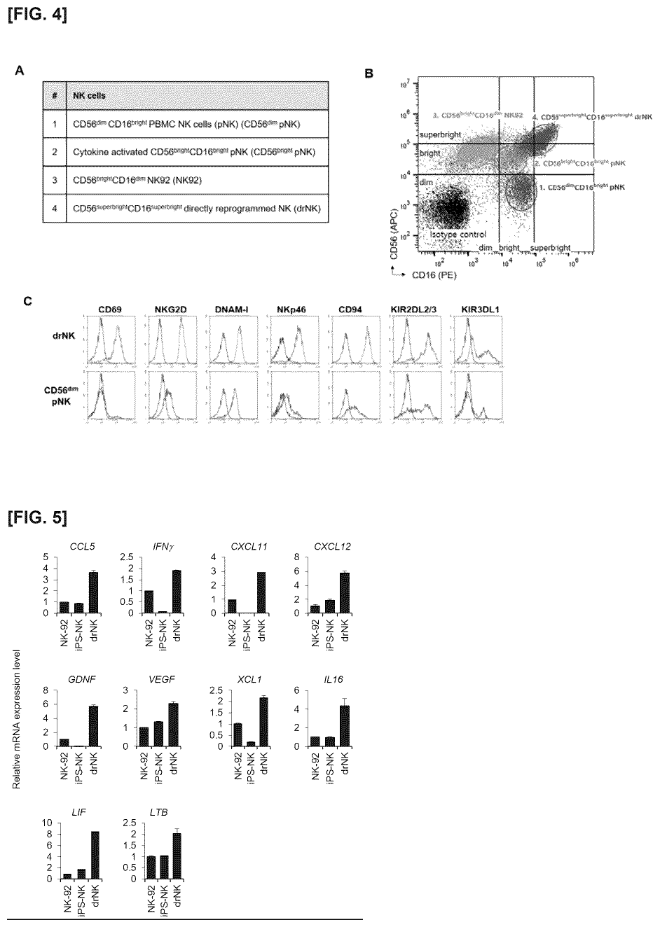

The gene expression levels of neurotrophic factors GDNF and IGFBP-2 were quantitatively analyzed by qPCR in hPSCs, the above-differentiated NCSCs, and the induced SCPs of the present invention, and it was confirmed that the levels were significantly higher in SCPs compared to NCSCs (A of FIG. 3). To obtain conditioned medium (CM) to comparatively analyze proteins secreted from NCSCs and induced SCPs that influence nerve regeneration and growth, 10⁵ SCP or NCSC cells were seeded into a 30 mm culture dish with 2 ml of culture medium. After 48 hours, the culture medium was filtered using a 0.22 μm filter (Millipore). To measure the concentration of secreted neurotrophic factor GDNF, ELISA was performed on the conditioned medium derived from SCPs and NCSCs according to the manufacturer's protocol (Abcam).

As a result, it was confirmed that SCPs secreted more GDNF (16.6 pg/ml) than NCSCs (3.3 pg/ml) (left of B of FIG. 3). To measure cytokine levels in NCSC-CM and SCP-CM, the Proteome Profiler Human XL Cytokine Array Kit (ARY022B; R&D Systems) was used according to the manufacturer's instructions. By obtaining images using an Amersham Imager 600 (GE Healthcare Life Sciences), and performing quantitative analysis using ImageJ (open source software), it was confirmed that SCPs secreted more factors [1150.5 MPD (Mean Pixel Density)] compared to NCSCs (157 MPD) (right of B of FIG. 3). Therefore, it can be inferred that the SCPs of the present invention exhibit greater efficacy on nerve growth and regeneration compared to NCSCs due to the higher secretion of neurotrophic factors.

### Example 4: Phenotypic Characterization of Directly Reprogrammed Natural Killer (drNK) Cells

To obtain induced drNK cells, PBMCs separated from human peripheral blood by Ficoll density gradient were transfected with the reprogramming factor OSKM, and the PBMCs were cultured together with polybrene (4 μg/ml) for 1 day. On the following day, 3×10⁵ of the transfected cells were seeded into 48-well culture plates and further cultured for 5 days in culture medium RIM (StemSpan SFEM II comprising 10% FBS, 1% Penicillin/Streptomycin, 5 μM CHIR99021, 20 ng/ml human IL-3,20 ng/ml human IL-6, 20 ng/ml human SCF, 20 ng/ml human FLT3L, and 20 ng/ml human TPO). The cells were then cultured for 18 to 40 days in culture medium RMM (StemSpan SFEM II comprising 10% FBS, 1% Penicillin/Streptomycin, 200 IU/ml human IL-2, 20 ng/ml human IL-7, 20 ng/ml human IL-15, 20 ng/ml human SCF, 20 ng/ml human FLT3L, and 2 μM StemRegenin I).

To confirm whether drNK cells were generated through the above direct reprogramming, the cells were stained with anti-CD56-APC (Biolegend) antibody and anti-CD16-PE (Biolegend) antibody as NK cell markers, and NK cell populations (CD56⁺ and CD16⁺) were analyzed using flow cytometry. As control cells, fresh primary NK cells isolated from PBMCs (pNK), pNK cells activated with 200 IU/ml human IL-2 and 20 ng/ml human IL-15 for 4 to 14 days (ApNK), and the NK cell line NK92 (ATCC) were used.

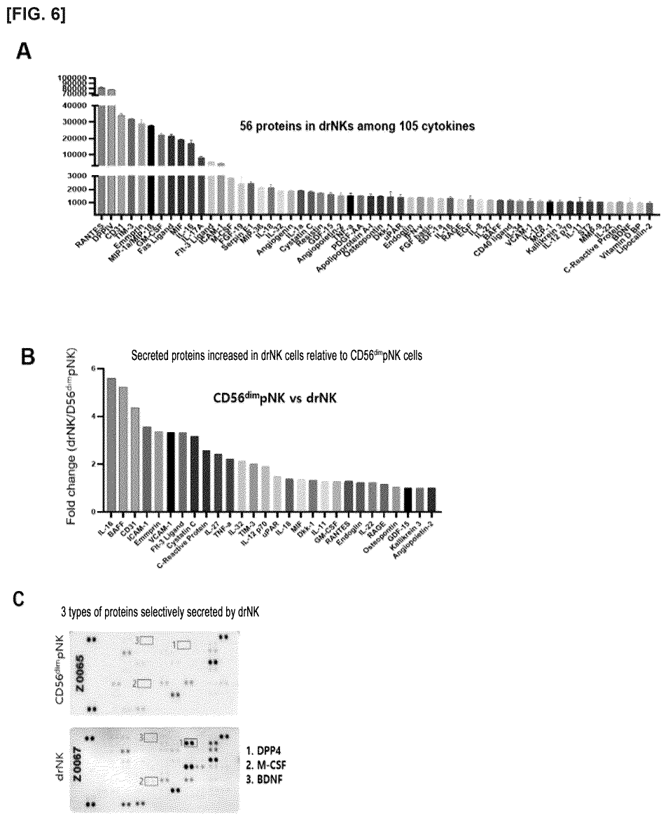

It was confirmed that according to the expression of CD56 and CD16 markers, the major cell populations were distinguished into the following cellular phenotypes: drNK: CD56^{superbright}CD16^{superbright}NK92: CD56^{bright}CD16^{dim}, pNK (CD56^{dim}pNK): CD56^{dim}CD16^{bright}, ApNK (CD56^{bright}pNK): CD56^{bright}CD16^{bright} (A and B of FIG. 4). The intensity was designated as dim (10⁴ or less), bright (10⁴ to 10⁵), and superbright (10⁵ or more) based on the fluorescence intensity of CD56 and CD16 of the most commonly used pNK (CD56^{dim}pNK). In particular, comparison of NK cell receptor expressions confirmed that the expressions of NK cell activating receptors such as CD69, NKG2D, DNAM-1, and NKp46 were higher in drNKs than in CD56^{dim}pNK (FIG. 4C).

### Example 5: Quantitative Analysis of Cytokine/Chemokine Gene Expression Levels in drNK Cells

For the production of iPSC-NK cells as a control for comparison with drNK, induced pluripotent stem cells (iPSCs) were dissociated into single cells by treatment with ReLeSR (Stem Cell Technologies). To STEMdiff APEL2 medium (Stem Cell Technologies), a medium composition for rotary embryoid bodies, was added 1×penicillin/streptomycin (Invitrogen), 40 ng/ml SCF (Invitrogen), 20 ng/ml VEGF (R&D), and 20 ng/ml BMP4 (R&D). The suspended cells at 3×10⁴ cells/ml were seeded to 3,000 cells per well in a round-bottom 96-well plate, centrifuged at 1,500 rpm for 4 minutes at 8° C., and cultured for 3 to 4 days in a 37° C. incubator, followed by half-medium changes with fresh medium and further culture for 9 to 11 days. On days 9 to 11 of rotary embryoid body differentiation, rotary embryoid bodies in wells 6 to 8 from the 96-well plate were transferred into well 1 of a 2% gelatin-coated 24-well plate. The differentiation culture medium comprised 85% DMEM/F12 (GIBCO), 15% FBS (GIBCO), 5 ng/ml sodium selenite (Sigma), 50 μM ethanolamine (Sigma), 20 μg/ml ascorbic acid (Sigma), 25 μM β-mercaptoethanol (GIBCO), 1×Glutamax (GIBCO), 1% penicillin/streptomycin (GIBCO), and cytokines including 5 ng/ml IL-3 (Peprotech), 10 ng/ml IL-15 (Peprotech), 20 ng/ml IL-7 (Peprotech), 20 ng/ml SCF (Invitrogen), and 10 ng/ml Flt3L (Peprotech). The cells were further cultured for 28 days in NK differentiation medium supplemented every 5 to 7 days with the above cytokines excluding IL-3, and differentiated into iPSC-NK cells. Pure iPSC-NK cells were isolated using an NK isolation kit (Miltenyi Biotec), and subsequently cultured in culture medium comprising 90% RPMI 1640, 10% FBS, 1% penicillin/streptomycin, 20 ng/ml IL-15, and 20 ng/ml IL-2.

qRT-PCR was performed to quantitatively analyze the gene expression of cytokines or chemokines expressed by the drNK compared to control NKs (NK-92 and iPS-NK cells), and it was confirmed that the expression in drNK relatively increased compared to the NK-92 and iPS-NK groups. When the expression level in NK-92 was set to 1, the fold expressions in iPS-NK and drNK were as follows: [CCL5: NK-92 (1), IPS-NK (0.85), drNK (3.64); IFN-γ: NK-92 (1), iPS-NK (0.1), drNK (1.9); CXCL11: NK-92 (1), iPS-NK (0.01), drNK (2.93); CXCL12: NK-92 (1), iPS-NK (1.82), drNK (5.73); GDNF: NK-92 (1), iPS-NK (0.03), drNK (5.69); VEGF: NK-92 (1), iPS-NK (1.29), drNK (2.3); XCL1: NK-92 (1), iPS-NK (0.18), drNK (2.15); IL16: NK-92 (1), iPS-NK (0.98), drNK (4.35); LIF: NK-92 (1), iPS-NK (1.78), drNK (8.5); LTB: NK-92 (1), iPS-NK (1.02), drNK (2.01)] (FIG. 5). Accordingly, by comparatively analyzing the expression levels of cytokine/chemokine target genes that may play an important role in NK function of NK-92, iPS-NK, and drNK cells, 10 genes (CCL5, IFN-γ, CXCL11, CXCL12, GDNF, VEGF, XCL1, IL16, LIF, and LTB) overexpressed in drNK cells were observed.

### Example 6: Identification of drNK-Secreted Factors Using Human Proteome Cytokine Array

The secreting ability of substances released by drNK cells that may influence neural regeneration, particularly cytokines, was analyzed in culture medium. Specifically, to obtain conditioned medium (CM) of NK cells, drNK cells and CD56^{dim}pNK cells were cultured in culture dishes at a density of 10⁶ cells/ml. After 24 hours, the culture medium was filtered using a 0.22 μm filter (Millipore). To measure cytokine levels in drNK-CM and CD56 ^{dim}pNK-CM, a Proteome Profiler Human XL Cytokine Array Kit (ARY022B; R&D Systems) was used according to the manufacturer's instructions. Quantitative analysis of the final images was performed using ImageJ software.

Through ImageJ analysis (threshold: image intensity of 1,000 Mean Pixel Density (MPD) or higher), 56 proteins secreted by drNK cells were identified [RANTES (average=81,174 MPD), DPPIV (average=78,046 MPD), CD31 (average=33,901 MPD), TIM-3 (average=31,688 MPD), Emmprin (average=29,204 MPD), MIP-1α/MIP-1β (average=27,937 MPD), GM-CSF (average=22,299 MPD), Fas Ligand (average=21,602 MPD), MIF (average=19,398 MPD), IL-16 (average=16,867 MPD), IL-17A (average=8,249 MPD), Flt-3 Ligand (average=5,834 MPD), ICAM-1 (average=4,532 MPD), M-CSF (average=2,867 MPD), FGF-19 (average=2,481 MPD), Serpin E1, MIP-3β (average=2,165 MPD), IL-18 (average=2,144 MPD), IL-32 (average=1,950 MPD), Angiogenin (average=1,901 MPD), IL-1α (average=1,895 MPD), Cystatin C (average=1,792 MPD), Resistin (average=1,699 MPD), GDF-15 (average=1,635 MPD), Angiopoietin-2 (average=1,558 MPD), TNF-α (average=1,546 MPD), PDGF-AA (average=1,536 MPD), Apolipoprotein A-I (average=1,507 MPD), Osteopontin (average=1,489 MPD), Dkk-1 (average=1,447 MPD), uPAR (average=1,422 MPD), Endoglin (average=1,411 MPD), IFN-γ (average=1,394 MPD), FGF basic (average=1,381 MPD), SDF-1α (CXCL12) (average=1,378 MPD), IL-1β (average=1,363 MPD), RAGE (average=1,327 MPD), EGF (average=1,277 MPD), IL-8 (average=1,252 MPD), IL-27 (average=1,208 MPD), BAFF (average=1,191 MPD), CD40 ligand (average=1,159 MPD), IL-34 (average=1,129 MPD), VCAM-1 (average=1,127 MPD), IL-1ra (average=1,113 MPD), MCP-1 (average=1,094 MPD), Kallikrein 3 (average=1,088 MPD), IL-12 p70 (average=1,088 MPD), IL-11 (average=1,087 MPD), ST2 (average=1,059 MPD), MMP-9 (average=1,058 MPD), IL-22 (average=1,057 MPD), C-Reactive Protein (average=1,041 MPD), BDNF (average=1,034 MPD), Vitamin D BP (average=1,032 MPD), and Lipocalin-2 (average=1,004 MPD)] (FIG. 6A).

In comparison with control CD56 ^{dim}pNK cells, 28 proteins were identified as being secreted at quantitatively higher levels in drNK cells [IL-16 (5.58-fold), BAFF (5.23-fold), CD31 (4.36-fold), ICAM-1 (3.57-fold), Emmprin (3.39-fold), VCAM-1 (3.33-fold), Flt-3 Ligand (3.32-fold), Cystatin C (3.16-fold), C-Reactive Protein (2.58-fold), IL-27 (2.44-fold), TNF-α (2.22-fold), IL-32 (2.16-fold), TIM-3 (2.01-fold), IL-12 p70 (1.94-fold), uPAR (1.53-fold), IL-18 Bpa (1.40-fold), MIF (1.39-fold), Dkk-1 (1.35-fold), IL-11 (1.33-fold), GM-CSF (1.30-fold), RANTES (1.29-fold), Endoglin (1.24-fold), IL-22 (1.16-fold), RAGE, Osteopontin (1.06-fold), GDF-15 (1.02-fold), Kallikrein 3 (1.02-fold), and Angiopoietin-2 (1.02-fold)] (B of FIG. 6).

Additionally, three proteins (DPP4, M-CSF, and BDNF) were identified as being specifically secreted by drNK cells (C of FIG. 6).

### Example 7: Confirmation of the Damaged Neuron Clearing Effect of drNK and its Dependence on CD16 Expression

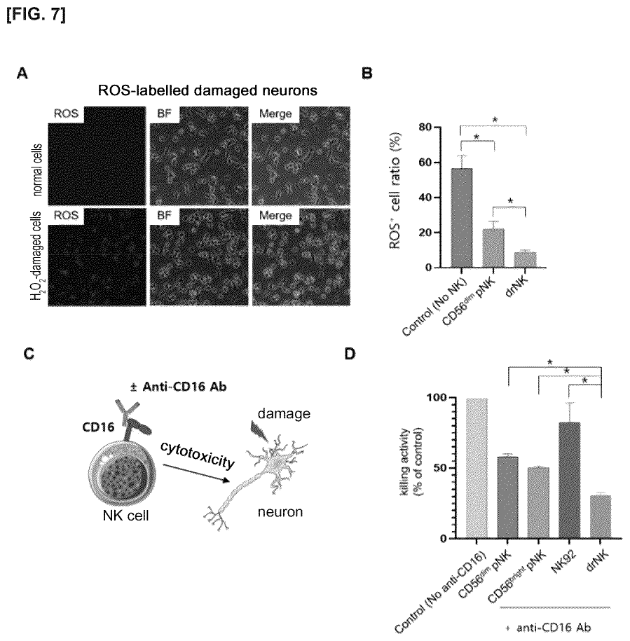

To evaluate the selective clearing effect of NK cells on damaged neurons, a partially damaged SH-SY5Y model was used. To induce damaged neuronal cells, SH-SY5Y cells were treated for 24 hours in DMEM/F12 (1:1) medium comprising 10% FBS and 200 μM H₂O₂, and it was confirmed that most of the cells were labeled as damaged cells by an ROS marker (MitoSox Red, Thermo Fisher Scientific) (A of FIG. 7). To induce partially damaged neuronal cells, SH-SY5Y cells were treated for 24 hours in DMEM/F12 (1:1) medium comprising 10% FBS and 200 μM H₂O₂. Untreated and H₂O₂-treated SH-SY5Y cells were mixed at a ratio of 1:3, plated, and on the following day, co-cultured with CD56^{dim}pNK or drNK cells (E: T=0.25:1) for 24 hours. The number of ROS-positive neuronal cells was then evaluated, and it was confirmed that, compared to the proportion of ROS-positive neuronal cells in the control group without NK cell co-culture (56.5%), the proportion was significantly reduced in CD56^{dim}pNK co-culture (22.0%) and in drNK co-culture (8.9%). Furthermore, drNK eliminated more than twice the number of damaged neuronal cells compared to CD56^{dim}pNK (B of FIG. 7).

To examine the correlation between the damaged neuron-clearing effect of natural killer (NK) cells and the expression level of CD16, neuronal injury was induced by treating SH-SY5Y neuronal cells, the damaged neuron model as described above, with H₂O₂, followed by co-culture with CD16 antibody and NK cells (E:T=1:1). After 4 hours, the damaged neuron-clearing effect of NK cells was analyzed. Specifically, SH-SY5Y cells were treated with 200 nM H₂O₂ in DMEM/F12 (1:1) medium comprising 10% FBS for 24 hours to induce neuronal damage. NK cells labeled with a cell tracker, damaged neurons, and CD16 antibody (BioLegend, used at a dilution of 1:20) were cultured either alone or together (E:T=1:1). After 4 hours, propidium iodide (PI, Thermo Fisher Scientific) staining was performed and analyzed by flow cytometry (C of FIG. 7).

With the damaged neuron-clearing effect in the absence of antibody set as the baseline (100%), the clearing effect was reduced to 58.1% for CD56^{dim}pNK, 50.1% for CD56^{bright}pNK, 82.6% for NK92, and 30.9% for drNK by CD16 antibody treatment. In conclusion, it was confirmed that the inhibitory effect of CD16 antibody is proportional to the level of CD16 expression, and that in the case of drNK cells of the present invention, the inhibitory effect of CD16 antibody was the greatest at 69.1% (D of FIG. 7). The list of antibodies used in the present invention is shown in Table 1 below.

TABLE 1

| Protein | Company (Cat. No.) | Dilution |
|---|---|---|
| SOX10 | Abcam (AB155279) | 1:200 |
| GAP43 | Abcam (AB75810) | 1:500 |
| TUJ1 | Abcam (AB78078) | 1:1000 |
| IGFBP2 | Cell signaling | 1:200 |
| | Technology (3922) | |
| S100B | Abcam (ab52642) | 1:1000 |
| MBP | Abcam (ab209328) | 1:1000 |
| CD16 | Biolegend (302008) | 1:20 |
| CD56 | Biolegend (318310) | 1:20 |
| CD69 | Biolegend (310906) | 1:20 |
| CD94 | Biolegend (305508) | 1:20 |
| NKG2D | Biolegend (320808) | 1:20 |
| NKp46 | Biolegend (331908) | 1:20 |
| DNAM-1 | Biolegend (338312) | 1:20 |
| KIR2DL2/3 | Biolegend (312604) | 1:20 |
| KIR3DL1 | Biolegend (312706) | 1:20 |

### Example 8: Evaluation of the Effect of SCP/SCP-SC and NK Cells on Neurite Outgrowth

To evaluate the effect of SCP/SCP-SC and NK cells on neurite outgrowth, a scratch model of neurons differentiated from hiPSCs and a partially damaged SH-SY5Y model were used (A of FIG.). First, iPSCs were cultured in 500×500 μm colony size in embryoid body (EB) culture medium (90% DMEM/F12 and 10% serum replacement comprising 1% penicillin/streptomycin, 1×MEM NEAA, 1×Glutamax, and 0.1 mM β-mercaptoethanol) under suspension for 7 days. Subsequently, the cells were transferred to neurosphere (NS) culture medium (DMEM/F12 comprising 1×N2, 1×B27, 1% penicillin/streptomycin, 20 ng/ml hEGF, 20 ng/ml bFGF, and 10 ng/ml hLIF), and cultured under rotary orbital conditions at 20 to 22 rpm. NS cells were subcultured every 5 to 7 days to 250×250 μm size, and during the second passage (NS p2), the NS p2s were attached onto cover glasses pre-coated with 0.01% poly-L-lysine (PLL) at 4° C. for 24 hours, and cultured in adherent NS culture medium [Neurobasal medium (Cat. No. 21103-049, GIBCO) comprising 1×N2, 1×B27, 1% penicillin/streptomycin, 1×Glutamax, 25 ng/ml BDNF, and 25 ng/ml GDNF] for 7 to 14 days until neurite outgrowth was observed.

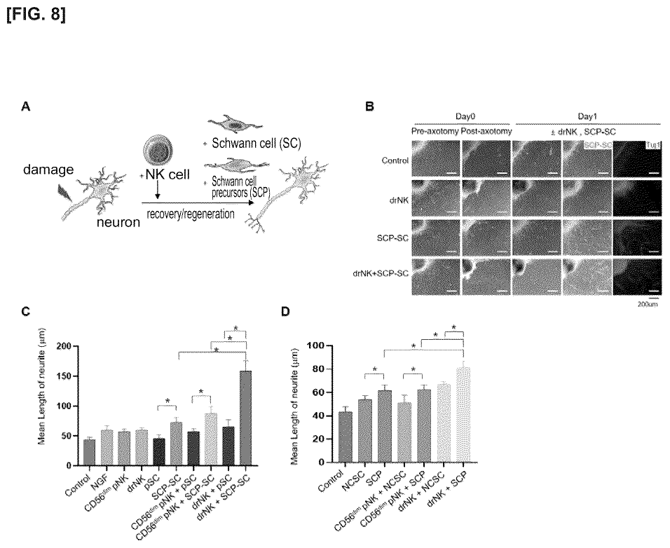

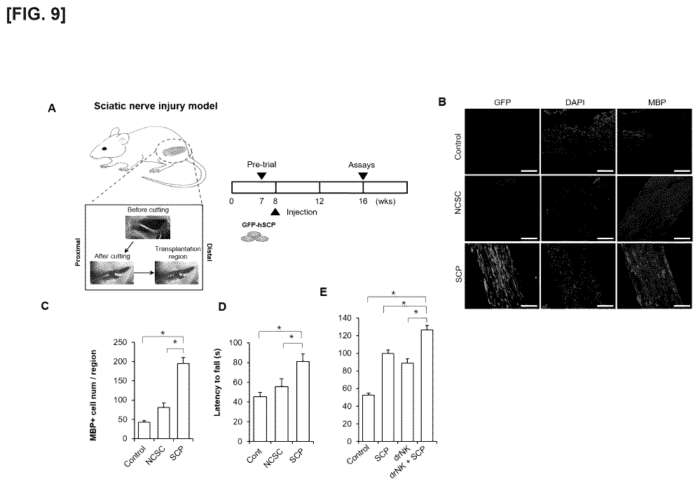

For neurite injury, neurites extending from the NS body were scratched and removed (damaged) using a sterile pipette tip. On the following day, SCs (10,000 cells/cm²) and drNK cells (10,000 cells/ml) were co-cultured. After 24 hours of co-culture, cell morphology was observed and immunocytochemical staining was performed using a βIII-tubulin antibody (Cat. No. ab78078, Abcam, 1:200 dilution). Through pre-and post-injury images and immunostaining after 24 hours of co-culture of SCP-SC (GFP-labeled) and drNK (which labelled neurites (TUJ1: red) and nuclei (DAPI: blue)), differences in neurite outgrowth across each experimental group were confirmed (FIG. 8B).

Using the same method as described above for B of FIG. 8, neurite outgrowth was quantitatively analyzed after treatment with either SCP-SC alone or a combination of SCP-SC+drNK followed by 48 hours of culture. Compared to the control group (average length=157.7 μm), the SCP-SC alone group (average length=251.7 μm) and the combined SCP-SC+drNK group (average length=315.9 μm) exhibited markedly enhanced neurite outgrowth (C of FIG. 8). In particular, it was confirmed that the combined SCP-SC+drNK group exhibited more than a 1.25-fold increase compared to the SCP-SC alone group.

To evaluate the effect of SCP/SCP-SC and NK cells on neurite outgrowth in a partial neuronal injury model, SH-SY5Y cells were treated for 24 hours in DMEM/F12 (1:1) medium comprising 10% FBS and 200 μM H₂O₂, as described in Example 7B. H₂O₂-treated SH-SY5Y cells and untreated cells were mixed at a 1:3 ratio, seeded at a density of 2×10³ cells/cm² onto Matrigel-coated plates in differentiation medium (DMEM/F12 (1:1) comprising 1 μM retinoic acid and 2% FBS), and cultured for 2 days in a 5% CO₂ incubator. After 2 days of culture, the differentiated SH-SY5Y cells were treated with NK, SCP, SCP-SC, NCSC, or NGF (Peprotech) [ratio=2 (neurons):1],further cultured for 2 days, and neurite outgrowth was analyzed. Images were acquired using an Axio Vert.A1 microscope (Carl Zeiss), and neurite length in each image was measured using AxioVs40 v4.8.2.0 software.

Compared to the control group (average length=43.5 μm), neurite outgrowth was promoted in the SCP-SC group (average length=72.9 μm), CD56^{dim}pNK group (average length=57.4 μm), drNK group (average length=59.8 μm), SCP-SC+CD56^{dim}pNK group (average length=87.3 μm), SCP-SC+drNK group (average length=158.6 μm), NGF group (average length=60.1 μm), NGF+drNK group (average length=67.2 μm), pSC group (average length=45.8 μm), pSC+CD56^{dim}pNK group (average length=57.1 μm), and pSC+drNK group (average length=65.1 μm) (D of FIG. 8). It was also confirmed that SCP-SC exhibited an effect more than 1.59-fold higher compared to pSC, drNK exhibited an effect more than 1.04-fold higher compared to CD56^{dim}pNK, and the combined SCP-SC+drNK group exhibited an effect more than 2.17-fold higher compared to the SCP-SC alone group.

When SCP and NCSC were treated in combination, the neurite outgrowth was as follows: SCP group (average length=71.1 μm), SCP+drNK group (average length=81.7 μm), SCP+CD56^{dim}pNK group (average length=53.7 μm), NCSC group (average length=53.7 μm), NCSC+drNK group (average length=67.3 μm), and NCSC+CD56^{dim}pNK group (average length=62.5 μm) (E of FIG. 8). Therefore, it was confirmed that SCP exhibited an effect more than 1.32-fold higher compared to NCSC, and the combined SCP+drNK group exhibited an effect more than 1.14-fold higher compared to the SCP alone group.

### Example 9: Promotion of Nerve Regeneration and Therapeutic Effect of Single or Combined Transplantation of SCP and NK Cells in a Sciatic Nerve Injury Animal Model

To analyze the effects on neuronal recovery and therapeutic efficacy of in vivo transplantation of Schwann cell precursors and natural killer cells, alone or in combination, a sciatic nerve injury mouse model was used (A of FIG. 9). First, the central region of the left sciatic nerve of 8-week-old C57BL/6 male mice was transected to create a 2 to 3 mm nerve defect. Schwann cell precursors differentiated in Example 1 and NCSCs differentiated in Example 3 were diluted in Matrigel (2×10⁴ cells/μl), and 1×10⁵ cells (5 μl of a cell suspension comprising SCPs labeled with GFP via lentiviral infection) were transplanted into the nerve defect site. In addition, the directly reprogrammed natural killer (drNK) cells (1×10⁷ cells/150 μl) were transplanted via the tail vein (A of FIG. 9). Sixteen weeks after transplantation, the sciatic nerves of each group were sampled, and immunohistochemical analysis was performed to confirm the neuronal regeneration and therapeutic effect of single NCSC transplantation and combined SCP transplantation.

It was confirmed by immunohistochemical analysis that the group transplanted with SCPs showed superior MBP-positive neuronal regeneration and therapeutic effect compared to the group transplanted with NCSCs. Quantitative analysis of the MBP-positive neurons revealed that the proportion of MBP-positive neurons was more than 2.1-fold higher in the SCP-transplanted group compared to the NCSC-transplanted group [control NCSC (80.6 cells/0.2 mm²), SCP of the present invention (195.3 cells/0.2 mm²)] (C of FIG. 9).

In the sciatic nerve injury model, a Rotarod test was performed 8 weeks after transplantation of NCSCs, SCPs, or a combination of SCPs and NK cells to evaluate motor function recovery. It was found that motor function was improved by more than 1.45-fold in the SCP transplantation group compared to the control NCSC group [control NCSC (55.6 sec), SCP of the present invention (80.94 sec)] (D of FIG. 9).

By the same method, analysis of the average motor function recovery using a Rotarod test revealed that the combined transplantation of SCPs with drNK exhibited more than a 1.26-fold superior therapeutic effect compared to SCPs alone [control SCP (100 sec), drNK (89.0 sec), SCP+drNK (126.6 sec)] (E of FIG. 9).

### Example 10: Promotion of Nerve Regeneration and Therapeutic Effect of Single or Combined Transplantation of SCP-SC and NK Cells

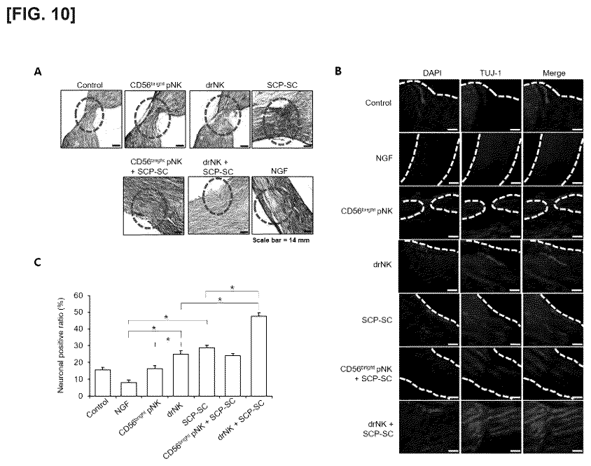

To analyze the neuronal recovery and therapeutic effects of a single or combined transplantation of nerve growth factor (NGF), Schwann cells, and natural killer (NK) cells, a partial sciatic nerve injury model was used. First, partial injury of the sciatic nerve was induced by pinching the central region of the sciatic nerve in 8-week-old immunodeficient Balb/c-nude male mice with forceps. At the injury site, either 5 μl of PBS comprising 20 μg/ml NGF (Peprotech) or 5 μl of a cell suspension prepared by diluting SCP-SCs in Matrigel (2×10⁴ cells/μl) was transplanted. In addition, depending on the experimental group, CD56^{bright}pNK or drNK cells (1×10⁷ cells) were transplanted into the tail vein as shown in FIG. 10A. Four weeks after transplantation, sciatic nerves from each group were sampled, and therapeutic efficacy for peripheral nerve disorders was confirmed morphologically by hematoxylin and eosin (H&E) staining. FIG. 10A shows representative staining results of each group (NGF, CD56^{bright}pNK, drNK, SCP-SC, CD56^{bright}pNK+SCP-SC, drNK+SCP-SC). Morphologically, compared to conventional CD56^{dim}pNK, the drNK of the present invention exhibited superior neuronal recovery and regeneration effects. Furthermore, compared to single treatment groups, combined SCP-SC+NK treatment groups exhibited relatively enhanced regenerative and therapeutic effects (FIG. 10A).

From the same samples as in A of FIG. 10, immunohistochemical analysis of neuronal marker TUJ1-positive cells revealed that compared to the control drug NGF treatment group, SCP-SC, CD56^{bright}pNK, and drNK single treatment groups exhibited relatively and significantly higher nerve regeneration-promoting effects [NGF (8.0%), SCP-SC (28.6%), CD56^{bright}pNK (16.3%), drNK (25%)]. It was also confirmed that the drNK of the present invention exhibited 1.53-fold greater nerve recovery and regeneration effect compared to conventional CD56^{bright}pNK. Moreover, the SCP-SC+drNK combined treatment group exhibited the most superior regenerative therapeutic effect compared to single transplantation. In particular, compared to the CD56^{bright}pNK+SCP-SC combined treatment group, the drNK+SCP-SC combined treatment group exhibited on average a 1.98-fold higher nerve regeneration effect [SCP-SC+drNK (47.6%), CD56^{bright}pNK+SCP-SC (24.0%)](FIGS. 10B and 10C).

To analyze the transplantation status and the degree of myelination in the drNK, SCP-SC, and drNK+SCP-SC groups in the sciatic nerve injury model shown in FIG. 10 above, immunohistochemical staining and qRT-PCR analysis of the Schwann cell marker S100 and the myelination marker MBP were performed 4 weeks post-transplantation. In the regeneration region of the combined transplantation group (drNK+SCP-SC), it was found that the protein and gene expression levels of S100 (A and B of FIG. 11) and MBP (C and D of FIG. 11) were significantly increased compared to those in the single transplantation groups [S100: Control (1), drNK (0.99), SCP-SC (1.38), drNK+SCP-SC (2.24); MBP: Control (1), drNK (1.09), SCP-SC (1.6), drNK+SCP-SC (2.2)]. In particular, comparison of the myelin-positive gene expression level showed that the combined transplantation group exhibited about 1.37-fold higher expression than the SCP-SC single transplantation group.

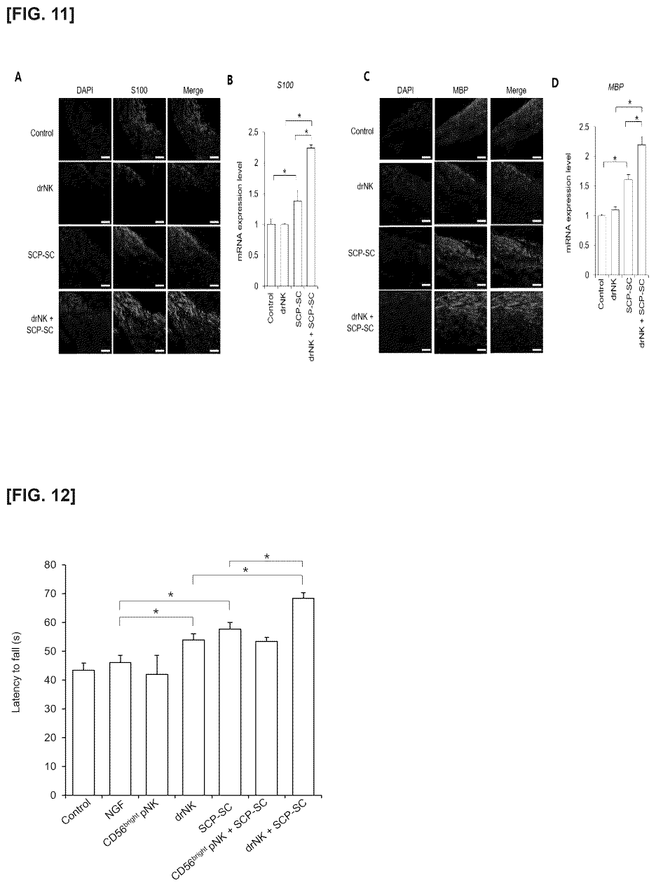

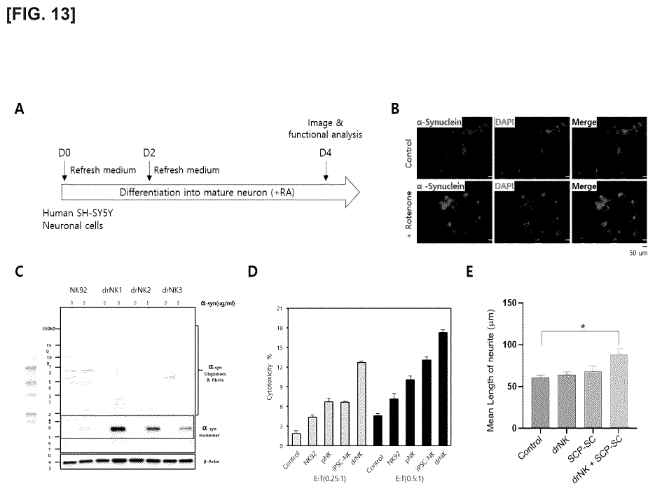

In addition, in the sciatic nerve injury model of FIG. 10, recovery of motor function associated with nerve regeneration was evaluated by Rotarod testing at 4 weeks post-transplantation. Among the single transplantation groups, the SCP-SC transplantation group exhibited the best motor function recovery (more than 1.25-fold compared to NGF), and the drNK transplantation group showed superior recovery compared to the CD56^{bright}pNK transplantation group [NGF (46 sec), CD56^{bright}pNK (42 sec), drNK (54 sec), SCP-SC (57.7 sec)]. Furthermore, among all experimental groups, the drNK+SCP-SC combined transplantation group exhibited the most enhanced motor function, while the CD56^{bright}pNK+SCP-SC combined transplantation group showed a relatively lower effect [drNK+SCP-SC (68.4 sec), CD56^{bright}pNK+SCP-SC (53.3 sec)]. Notably, compared to the SCP-SC single transplantation group, which showed the greatest effect among the single transplantation groups, the drNK+SCP-SC combined transplantation group demonstrated a statistically significant improvement of more than 1.18-fold in motor function recovery (FIG. 12).

### Example 11: Therapeutic Effect of Treatment With NK Cells and Schwann Cells, Alone or in Combination, in a Parkinson's Disease (PD) Cell Model

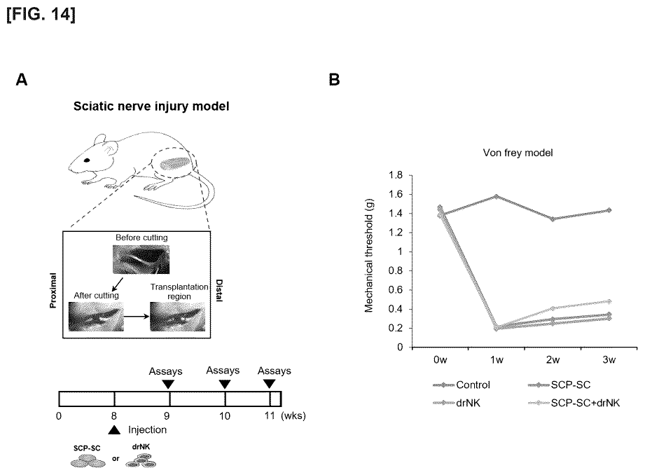

SH-SY5Y neuronal cells were induced to differentiate into cholinergic neurons by culturing for 4 days in DMEM/F12 medium (Thermo Fisher Scientific) comprising 1% FBS (Thermo Fisher Scientific), 10 μM retinoic acid (Sigma), and 1% P/S (Thermo Fisher Scientific). To model Parkinson's disease, the cells were treated with 10 μM rotenone (Sigma, Cat. no. 557368) for 24 hours (A of FIG. 13). The expression level of α-synuclein in the cells was compared between the control group and the rotenone-treated group by immunofluorescence staining. The cells were treated with 4% paraformaldehyde solution at room temperature for 10 minutes, washed with PBS, then treated for 30 minutes with PBS (staining solution) comprising 0.3% Triton X-100 (Sigma), 10% FBS (Thermo Fisher Scientific), and 1% BSA (Thermo Fisher Scientific). The staining solution was then incubated at 4° C. for 16 hours with an α-synuclein-specific antibody (Invitrogen, Cat. no. PA5-85343, dilution 1:500), washed, and subsequently reacted at room temperature for 20 minutes with a staining solution comprising Alexar-594 goat-anti-rabbit IgG (Thermo Fisher Scientific, dilution 1:200), followed by three washes with PBS. Nuclear staining was performed by treating with PBS comprising 300 nM DAPI (Millipore) for 1 minute, followed by washing. Fluorescence microscopy analysis revealed that the expression of α-synuclein was elevated in the rotenone-treated group compared to the control group (B of FIG. 13).

The endocytosis of α-synuclein by drNK cells and conventional NK cells (NK92) was examined using immunoblot analysis. 1×10⁶ NK92 cells and three different preparations of 1×10⁶ drNK cells derived from different donors were each incubated with 5 μg of α-synuclein aggregate protein in a 1 ml tube for 1 hour. After incubation, the cells were washed three times with PBS by centrifugation at 500×g for 3 minutes at 4° C. To the resulting cell pellets were added 1X Laemmli sample buffer (Bio-Rad, Cat. no. 1610747) and heated at 99° C. for 10 minutes. Each sample was then loaded onto a 4 to 12% SDS-PAGE gel (Bio-Rad, Cat. no. 4561095) and the separated proteins were blotted on a PVDF membrane (Millipore). The membranes were blocked with TBST solution containing 5% non-fat milk at room temperature for 1 hour and then incubated with an α-synuclein-specific antibody (Invitrogen, Cat. no. PA5-85343, dilution 1:1000) at 4° C. for 16 hours. Following three washes with TBST (10 minutes each), the membranes were incubated with a rabbit-HRP-labelled secondary antibody (Santa Cruz, dilution 1:5000) at room temperature for 1 hour, washed three times with TBST (10 minutes each), and then treated with ECL solution (Thermo) for protein expression analysis. The results demonstrated that all three types of drNK cells exhibited higher uptake of α-synuclein compared to NK92 cells (C of FIG. 13).

The cytotoxic activity of conventional NK cells (NK92, pNK, and iPSC-NK) and co-culture with drNK cells against the Parkinson's disease (PD) cell model were analyzed. Specifically, the differentiated SH-SY5Y cells were treated with 10 μM rotenone in DMEM/F12 (1:1) medium comprising 1% FBS for 24 hours to induce PD cells. The damaged neurons were then co-cultured with CellTracker-labeled NK cells for 16 hours (E:T=0.25:1, E:T=0.5:1), and cytotoxicity was assessed by propidium iodide (PI) staining followed by flow cytometry (D of FIG. 13). An E:T ratio of 0.25:1 exhibited cytotoxicity (%) as follows: Control (1.9%), NK92 (4.4%), pNK (6.76%), iPSC-NK (6.66%), and drNK (12.7%); and an E:T ratio of 0.5:1, exhibited cytotoxicity (%) as follows: Control (4.6%), NK92 (7.0%), pNK (10.0%), iPSC-NK (13.06%), and drNK (17.3%). These results demonstrate that drNK cells exhibit superior cytotoxic activity compared to other NK cell groups.

To induce PD neuronal cells, SH-SY5Y cells were treated with 10 μM rotenone in DMEM/F12 (1:1) medium comprising 1% FBS for 24 hours, seeded at 2×10³ cells/cm² onto Matrigel-coated plates in differentiation medium (DMEM/F12 (1:1) comprising 1 μM retinoic acid and 1% FBS), and incubated for 2 days in a 5% CO₂incubator. After 2 days of culturing, the differentiated SH-SY5Y cells were further treated with drNK, SCP-SC, or a combination of drNK and SCP-SC (ratio=2 (neurons): 1 (NK or SCP-SC)) and cultured for an additional 2 days. Neurite outgrowth was analyzed by measuring the length of neurites in each image using AxioVs40 v4.8.2.0 software. The mean neurite lengths were as follows: Control (60.8 μm), drNK (64.0 μm), SCP-SC (67.6 μm), and SCP-SC+drNK (88.8 μm) (E of FIG. 13).

### Example 12: Evaluation of Pain Alleviation Effect in a Sciatic Nerve Injury Pain Mouse Model

In a peripheral nerve injury mouse model induced by sciatic nerve damage, the effect of transplantation of SCP-derived Schwann cells (SCP-SCs) and directly reprogrammed natural killer cells (drNKs), either alone or in combination, on nerve regeneration and sensory function recovery was evaluated through behavioral analysis. For this purpose, 8-week-old immunodeficient male Balb/c-nude mice were used. After anesthetizing the mice with an anesthetic for animals, the thigh region was incised to expose the sciatic nerve. The central portion of the sciatic nerve was then completely transected using fine micro-scissors, and the gap between the severed nerve stumps was artificially maintained to establish a defect environment requiring nerve regeneration. Immediately after the injury, cells were transplanted according to the treatment groups as follows. In the SCP-SC single transplantation group, 5 μL of a Matrigel suspension containing SCP-derived Schwann cells at a concentration of 2×10⁴ cells/μL was locally injected into the injury site. In the drNK single transplantation group, drNK cells (1×10⁷ cells) suspended in a saline solution were administered systemically via intravenous injection into the tail vein. In the SCP-SC+drNK combination group, both cells were transplanted and injected under the same conditions, respectively (A of FIG. 14). To evaluate the recovery of sensory function following cell transplantation, a von Frey filament-based mechanical stimulation response test (von Frey test) was performed. This test was conducted by applying nylon filaments of a defined force to the plantar surface and measuring the mechanical pain threshold based on the observed behavioral responses (e.g., paw withdrawal, flinching, etc.). At week 3, the mechanical thresholds were 1.43 g in the control group, 0.30 g in the drNK-treated group, 0.34 g in the SCP-SC-treated group, and 0.48 g in the SCP-SC+drNK combination group (B of FIG. 14). Compared to the control group, both the drNK and SCP-SC single transplantation groups showed improved sensory function recovery, while the combination transplantation group demonstrated the highest improvement in mechanical pain threshold.

From the foregoing description, those skilled in the art to which the present invention pertains will be able to understand that the present invention may be embodied in other specific forms without modifying the technical concepts or essential characteristics thereof. In this regard, the embodiments described above should be understood to be illustrative rather than restrictive in every respect. The scope of the present invention should be construed as the meaning and scope of the appended claims rather than the detailed description, and all changes or variations derived from the equivalent concepts fall within the scope of the present invention.

1. A pharmaceutical composition for preventing or treating a neurological disease, comprising Schwann cell precursors (SCPs) or Schwann cells (SCs) differentiated therefrom; and natural killer (NK) cells as active ingredients, wherein:
the Schwann cell precursors (SCPs) express one or more selected from the group consisting of GAP43, SOX10, IGFBP2, and a combination thereof;
the Schwann cells (SCs) express one or more selected from the group consisting of S100B, SOX10, and a combination thereof; and
the natural killer (NK) cells express one or more selected from the group consisting of CD56⁺, CD16⁺, and a combination thereof.

2. The pharmaceutical composition according to claim 1, wherein the SCPs are prepared by a method of preparing SCPs from pluripotent stem cells, comprising:
(a) culturing pluripotent stem cells in a first medium comprising SB431542 and CT99021; and
(b) culturing the cells cultured in (a) in a second medium that further comprises Neuregulin-1 (NRG1).

3. The pharmaceutical composition according to claim 1, wherein the first medium of (a) can further comprise FGF2, and the second medium of (b) further comprises Stem Regenin 1.

4. The pharmaceutical composition according to claim 1, wherein the SCs are prepared by a method of preparing SCs from pluripotent stem cells, comprising:
(a) culturing pluripotent stem cells in a first medium comprising SB431542 and CT99021;
(b) culturing the cells cultured in (a) in a second medium that further comprises Neuregulin-1 (NRG1);
(c) recovering SCPs from the cultured medium; and
(d) culturing the recovered SCP in a third medium comprising FBS and NRG1.

5. The pharmaceutical composition according to claim 4, wherein the third medium of (d) further comprises one or more selected from the group consisting of retinoic acid, forskolin, and PDGF-BB.

6. The pharmaceutical composition according to claim 1, wherein the natural killer (NK) cells express one or more selected from the group consisting of CD56^{dim}, CD56^{bright}, CD56^{superbright}, CD16^{dim}, CD16^{bright}, CD16^{superbright}, and a combination thereof.

7. The pharmaceutical composition according to claim 1, wherein the NK cells are prepared by a method of preparing directly reprogrammed natural killer (drNK) cells from isolated cells, comprising:
(a) introducing a reprogramming factor into the isolated cells;
(b) from the day after introducing the reprogramming factor, culturing the cells in (a) i) in a first medium comprising cytokines, growth factors, and glycogen synthase kinase 3β (GSK3β) inhibitors to increase the efficiency of direct reprogramming, and ii) in a second medium comprising cytokines, growth factors, and aryl hydrocarbon receptor (AHR) inhibitors to promote the production of natural killer cells.

8. The pharmaceutical composition according to claim 7, wherein the directly reprogrammed natural killer (drNK) cells overexpress one or more genes selected from the group consisting of CCL5, IFN-γ, CXCL11, CXCL12, GDNF, VEGF, XCL1, IL16, LIF, and LTB, compared to a control group.

9. The pharmaceutical composition according to claim 6, wherein the directly reprogrammed natural killer (drNK) cells express one or more proteins selected from the group consisting of DPP4, M-CSF, and BDNF.

10. The pharmaceutical composition according to claim 1, wherein the neurological disease is one or more diseases selected from the group consisting of brain tumor, cerebral infarction, hypertensive cerebral hemorrhage, cerebral contusion, cerebral arteriovenous malformation, brain abscess, encephalitis, hydrocephalus, epilepsy, concussion, cerebral palsy, mild cognitive impairment, dementia, spinal cord tumor, spinal arteriovenous malformation, spinal cord infarction, pain, headache, migraine, Alzheimer's disease, Parkinson's disease, Huntington's disease, amyotrophic lateral sclerosis (ALS), Batten disease, Kearns-Sayre syndrome (KSS), chronic progressive external ophthalmoplegia (CPEO), mitochondrial encephalomyopathy, lactic acidosis, and stroke-like episodes (MELAS syndrome), myoclonic epilepsy with ragged-red fibers (MERRF) syndrome, neurogenic weakness with ataxia and retinitis pigmentosa (NARP) syndrome, Leigh syndrome, mitochondrial recessive ataxia syndrome (MIRAS), degenerative neurological diseases, schizophrenia, schizophreniform disorder, attention deficit hyperactivity disorder (ADHD), personality disorders, autism, post-traumatic stress disorder (PTSD), anxiety disorders, panic disorders, depression, chronic stress-related depression, delusional disorders, obsessive-compulsive disorders, anorexia nervosa, bulimia nervosa, obesity, cerebral ischemic diseases, neurodegenerative diseases, diabetic neuropathy, traumatic nerve injury, neurodegenerative disorders, neuropathic pain, epilepsy, chronic neuropathic pain, Guillain-Barré syndrome, myasthenia gravis, Rett syndrome, central sleep apnea, peripheral neuropathy, Charcot-Marie-Tooth disease, spinal muscular atrophy (SMA), autoimmune encephalitis, chronic traumatic encephalopathy (CTE), myotonic dystrophy, multiple sclerosis, schwannoma, neurofibromatosis, chronic inflammatory demyelinating polyneuropathy (CIDP), polyneuropathy, and neurinoma.

11. A cell therapy agent for preventing or treating a neurological disease, comprising Schwann cell precursors (SCPs) or Schwann cells (SCs) differentiated therefrom; and directly reprogrammed natural killer (drNK) cells as active ingredients, wherein:
the Schwann cell precursors (SCPs) express one or more selected from the group consisting of GAP43, SOX10, IGFBP2, and a combination thereof;
the Schwann cells (SCs) express one or more selected from the group consisting of S100B, SOX10, and a combination thereof; and
the natural killer (NK) cells express one or more selected from the group consisting of CD56⁺, CD16⁺, and a combination thereof.

12. The cell therapy agent according to claim 11, wherein the natural killer (NK) cells express one or more selected from the group consisting of CD56^{dim}, CD56^{bright}, CD56^{superbright}, CD16^{dim}, CD16^{bright}, CD16^{superbright}, and a combination thereof.

13. A method for preventing or treating a neurological disease, comprising administering to a subject in need thereof an effective amount of Schwann cell precursors (SCPs) or Schwann cells (SCs) differentiated therefrom, and natural killer (NK) cells, wherein:
the Schwann cell precursors (SCPs) express one or more selected from the group consisting of GAP43, SOX10, IGFBP2, and a combination thereof;
the Schwann cells (SCs) express one or more selected from the group consisting of S100B, SOX10, and a combination thereof; and
the natural killer (NK) cells express one or more selected from the group consisting of CD56⁺, CD16⁺, and a combination thereof.

14. The method according to claim 13, wherein the SCPs are prepared by a method of preparing SCPs from pluripotent stem cells, comprising:
(a) culturing pluripotent stem cells in a first medium comprising SB431542 and CT99021; and
(b) culturing the cells cultured in (a) in a second medium that further comprises Neuregulin-1 (NRG1).

15. The method according to claim 13, wherein the SCs are prepared by a method of preparing SCs from pluripotent stem cells, comprising:
(a) culturing pluripotent stem cells in a first medium comprising SB431542 and CT99021;
(b) culturing the cells cultured in (a) in a second medium that further comprises Neuregulin-1 (NRG1);
(c) recovering SCPs from the cultured medium; and
(d) culturing the recovered SCP in a third medium comprising FBS and NRG1.

16. The method according to claim 13, wherein the natural killer (NK) cells are directly reprogrammed natural killer (drNK) cells prepared by a method comprising:
(a) introducing a reprogramming factor into isolated cells; and
(b) from the day after introducing the reprogramming factor, culturing the cells in (a) i) in a first medium comprising cytokines, growth factors, and glycogen synthase kinase 3β (GSK3β) inhibitors to increase the efficiency of direct reprogramming, and ii) in a second medium comprising cytokines, growth factors, and aryl hydrocarbon receptor (AHR) inhibitors to promote the production of natural killer cells.

17. The method according to claim 13, wherein the neurological disease is selected from the group consisting of Alzheimer's disease, Parkinson's disease, amyotrophic lateral sclerosis (ALS), Huntington's disease, traumatic nerve injury, diabetic neuropathy, peripheral neuropathy, multiple sclerosis, and spinal cord injury.

18. The method according to claim 13, wherein the pharmaceutical composition is administered by a route selected from the group consisting of intravenous, intraperitoneal, intramuscular, subcutaneous, intradermal, intracerebrovascular, and intranasal administration.


SEQUENCE LISTING (40 sequences)

SEQ ID NO: 1 - DNA, 20 bp, synthetic construct
  source 1..20: mol_type = other DNA
ccagcagtcgtctttgtcac

SEQ ID NO: 2 - DNA, 20 bp, synthetic construct
  source 1..20: mol_type = other DNA
ctctgggttggcacacactt

SEQ ID NO: 3 - DNA, 20 bp, synthetic construct
  source 1..20: mol_type = other DNA
cacaacacgctgttcggcta

SEQ ID NO: 4 - DNA, 22 bp, synthetic construct
  source 1..22: mol_type = other DNA
cgatcctgcatctgtaaatcgc

SEQ ID NO: 5 - DNA, 20 bp, synthetic construct
  source 1..20: mol_type = other DNA
gacgctgtctttgcataggc

SEQ ID NO: 6 - DNA, 23 bp, synthetic construct
  source 1..23: mol_type = other DNA
ggatttaggcatcgttgtccttt

SEQ ID NO: 7 - DNA, 23 bp, synthetic construct
  source 1..23: mol_type = other DNA
attctcaacactccaaactgtgc

SEQ ID NO: 8 - DNA, 21 bp, synthetic construct
  source 1..21: mol_type = other DNA
actttagcttcgggtcaatgc

SEQ ID NO: 9 - DNA, 20 bp, synthetic construct
  source 1..20: mol_type = other DNA
ggccgcaaccaaaattcagg

SEQ ID NO: 10 - DNA, 19 bp, synthetic construct
  source 1..19: mol_type = other DNA
cggcagtagtggtgccttc

SEQ ID NO: 11 - DNA, 22 bp, synthetic construct
  source 1..22: mol_type = other DNA
acaactttggtatcgtggaagg

SEQ ID NO: 12 - DNA, 19 bp, synthetic construct
  source 1..19: mol_type = other DNA
gccatcacgccacagtttc

SEQ ID NO: 13 - DNA, 21 bp, synthetic construct
  source 1..21: mol_type = other DNA
ggcagtgcttcctagaagaga

SEQ ID NO: 14 - DNA, 21 bp, synthetic construct
  source 1..21: mol_type = other DNA
aagacacaaccccggtttttg

SEQ ID NO: 15 - DNA, 21 bp, synthetic construct
  source 1..21: mol_type = other DNA
gacccaggtctacgatgggaa

SEQ ID NO: 16 - DNA, 20 bp, synthetic construct
  source 1..20: mol_type = other DNA
gtgagctgagtcaagcggag

SEQ ID NO: 17 - DNA, 20 bp, synthetic construct
  source 1..20: mol_type = other DNA
gtcgccagcagctaaaacag

SEQ ID NO: 18 - DNA, 20 bp, synthetic construct
  source 1..20: mol_type = other DNA
actgggatgctcttcgacct

SEQ ID NO: 19 - DNA, 21 bp, synthetic construct
  source 1..21: mol_type = other DNA
gacaatggcgatgaccactca

SEQ ID NO: 20 - DNA, 21 bp, synthetic construct
  source 1..21: mol_type = other DNA
cagctccttcatacccgactt

SEQ ID NO: 21 - DNA, 23 bp, synthetic construct
  source 1..23: mol_type = other DNA
acgaagctacttgacgaaaagac

SEQ ID NO: 22 - DNA, 22 bp, synthetic construct
  source 1..22: mol_type = other DNA
gtttcagcagaaccatttgcag

SEQ ID NO: 23 - DNA, 19 bp, synthetic construct
  source 1..19: mol_type = other DNA
ccaacgtgacggacttccc

SEQ ID NO: 24 - DNA, 21 bp, synthetic construct
  source 1..21: mol_type = other DNA
tacacgactatgcggtacagc

SEQ ID NO: 25 - DNA, 20 bp, synthetic construct
  source 1..20: mol_type = other DNA
gtacgggcctctctggtaca

SEQ ID NO: 26 - DNA, 20 bp, synthetic construct
  source 1..20: mol_type = other DNA
gtccaccatatcggggtgac

SEQ ID NO: 27 - DNA, 20 bp, synthetic construct
  source 1..20: mol_type = other DNA
aagtgccaactaggtacggg

SEQ ID NO: 28 - DNA, 20 bp, synthetic construct
  source 1..20: mol_type = other DNA
catagcactgagcctcctct

SEQ ID NO: 29 - DNA, 21 bp, synthetic construct
  source 1..21: mol_type = other DNA
tggcctacatagccttcaaga

SEQ ID NO: 30 - DNA, 19 bp, synthetic construct
  source 1..19: mol_type = other DNA
gagatgccactgtcgctgt

SEQ ID NO: 31 - DNA, 19 bp, synthetic construct
  source 1..19: mol_type = other DNA
acctatgccctgaccgttg

SEQ ID NO: 32 - DNA, 21 bp, synthetic construct
  source 1..21: mol_type = other DNA
tgctggggaaggcaatagact

SEQ ID NO: 33 - DNA, 21 bp, synthetic construct
  source 1..21: mol_type = other DNA
gaccctcatcaacgtgttcca

SEQ ID NO: 34 - DNA, 21 bp, synthetic construct
  source 1..21: mol_type = other DNA
ccacaagcaccacatactcct

SEQ ID NO: 35 - DNA, 20 bp, synthetic construct
  source 1..20: mol_type = other DNA
cctcacagatcgcctacacc

SEQ ID NO: 36 - DNA, 23 bp, synthetic construct
  source 1..23: mol_type = other DNA
catataggagaaggccgagtaga

SEQ ID NO: 37 - DNA, 21 bp, synthetic construct
  source 1..21: mol_type = other DNA
agggcagaatcatcagcaagt

SEQ ID NO: 38 - DNA, 20 bp, synthetic construct
  source 1..20: mol_type = other DNA
agggtctcgattggatggca

SEQ ID NO: 39 - DNA, 22 bp, synthetic construct
  source 1..22: mol_type = other DNA
tggaagagattctggctagtgt

SEQ ID NO: 40 - DNA, 20 bp, synthetic construct
  source 1..20: mol_type = other DNA
ggaccctcagaatgggaacc